(19) United States
(12) Patent Application Publication
(10) Pub. No.: US 2026/0207976 A1
(43) Pub. Date: Jul. 23, 2026

(54) SCREENING, MONITORING, AND TREATMENT FRAMEWORK FOR FOCUSED ULTRASOUND

(71) Applicant: West Virginia University Board of Governors on behalf of West Virginia University, Morgantown, WV (US)

(72) Inventors: Ali Rezai, Morgantown, WV (US); Victor Finomore, Morgantown, WV (US); James Mahoney, Morgantown, WV (US); Clay Marsh, Morgantown, WV (US); Jeff Carpenter, Morgantown, WV (US); Pierre D'haese, Morgantown, WV (US); Marc Haut, Morgantown, WV (US); Annahita Amireskandari, Morgantown, WV (US)

(21) Appl. No.: 19/564,502

(22) Filed: Mar. 12, 2026

Related U.S. Application Data
(63) Continuation-in-part of application No. 18/310,465, filed on May 1, 2023.
(60) Provisional application No. 63/337,146, filed on May 1, 2022.
(60) Provisional application No. 63/348,713, filed on Jun. 3, 2022.
(60) Provisional application No. 63/392,572, filed on Jul. 27, 2022.
(60) Provisional application No. 63/400,960, filed on Aug. 25, 2022.
(60) Provisional application No. 63/435,456, filed on Dec. 27, 2022.

(51) Int. Cl. A61N 7/02; A61N 7/00

(52) U.S. Cl. CPC A61N 7/02; A61N 2007/0026

(57) ABSTRACT

A method of improving a visuospatial impairment in a patient suffering from Parkinson's Disease is provided by delivering a focused ultrasound signal to a neural target site of the patient. The patient's visual perception, blurry vision, judging distance, depth perception, and/or another visual and spatial deficit can be improved. The neural target site includes the subthalamic nucleus, the globus pallidus, the putamen, the nucleus accumbens, the ventral striatum, the ventral internal capsule, a thalamic relay nucleus, and/or combinations thereof.

### RELATED APPLICATIONS

This application is a continuation-in-part of U.S. application Ser. No. 18/310,465 filed on May 1, 2023, which claims priority from each of U.S. Application No. 63/337,146, filed 1 May 2022, U.S. Application No. 63/348,713, filed 3 Jun. 2022, U.S. Application No. 63/392,572, filed 27 Jul. 2022, U.S. Application No. 63/400,960, filed 25 Aug. 2022, U.S. Application No. 63/435,456, filed 27 Dec. 2022. The subject matter of all above-referenced applications is incorporated herein by reference in their entirety.

### TECHNICAL FIELD

This invention relates generally to assisted decision making systems and more specifically to a framework for focused ultrasound treatment.

### BACKGROUND

Neuromodulation also refers to an emerging class of medical therapies that target the nervous system for restoration of function, relief of pain, or control of symptoms. The therapies consist primarily of targeted stimulation by various forms of energy. Electrical stimulation devices include deep brain stimulation systems, spinal cord stimulators, vagus nerve stimulators, and transcutaneous electrical nerve stimulation devices. Other methods for neuromodulation can use magnetic stimulation, such as transcutaneous magnetic stimulation, as well as sound, in focused ultrasound systems. Neuromodulation appears promising in treating a number of disorders, but appropriate timing, dosage, and location of neuromodulation can be difficult to determine for a given disorder.

### SUMMARY

In accordance with one example, a method is disclosed for providing focused ultrasound treatment. A patient is screened to determine if focused ultrasound treatment is appropriate for the patient to treat a disorder. The patient is monitored to measure a plurality of wellness-related parameters for the patient and detect or predict an onset of symptoms associated with the disorder from the plurality of wellness-related parameters if focused ultrasound treatment has been determined to be appropriate for the patient. A personalized location for focused ultrasound treatment is determined for the patient according to at least one of the plurality of wellness-related parameters. At least one parameter associated with the focused ultrasound treatment is selected according to at least one of the plurality of wellness-related parameters. Focused ultrasound treatment is provided to the patient at the selected location using the selected at least one parameter. The plurality of wellness-related parameters are measured after focused ultrasound treatment is provided to determine an effectiveness of the focused ultrasound treatment.

In accordance with another example, a system is provided for generating a clinical parameter for a user. A physiological sensing device monitors a first plurality of wellness-relevant parameters representing the user over a defined period. A portable computing device obtains a second plurality of wellness-relevant parameters representing the user via a portable computing device. A network interface retrieves a third plurality of wellness-relevant parameters representing the user from an electronic health records (EHR) system, the first plurality of wellness-relevant parameters, the second plurality of wellness-relevant parameters, and the third plurality of wellness-relevant parameters collectively forming a set of wellness-relevant parameters. A feature aggregator generates a set of aggregate parameters from set of wellness-relevant parameters, with each of the set of aggregate parameters comprising a unique proper subset of the set of wellness-relevant parameters. A predictive model assigns the clinical parameter to the user according to a subset of the set of aggregate parameters.

In accordance with a further example, a method is provided for generating a value representing one of a risk and a progression of a disorder. A first image, representing a brain of a patient, is acquired from a first imaging system, and a second image, representing one of a retina, an optic nerve, and a vasculature associated with one of the optic nerve and the retina of the patient, is acquired from a second imaging system. A representation of each of the first image and the second image are provided to a machine learning model. The value is generated at the machine learning model from the representation of the first image and the representation of the second image, and the patient is assigned to one of a plurality of intervention classes according to the generated value.

In accordance with a still further example, a method is provided for determining a risk of a disorder from imaging of a brain of a patient. A first image, representing a structure of the brain, is acquired from a first imaging system, and a second image, representing a connectivity of the brain, is acquired from one of the first imaging system and a second imaging system. The first image is segmented into a plurality of subregions of the brain to generate a segmented first image, such that each of at least a subset of a plurality of voxels comprising the first image are associated with one of the plurality of subregions. A representation of the segmented first image and the second image are provided to a machine learning model trained on imaging data for a plurality of patients having known outcomes. A clinical parameter representing the risk of the patient for the disorder is generated from the representation of the segmented first image and the second image.

In accordance with another example, a method of improving a visuospatial impairment in a patient suffering from Parkinson's Disease is provided that comprises delivering a focused ultrasound signal to a neural target site of the patient. The improvement in visuospatial impairment can comprise improving the visual perception, the blurry vision, the judging distance, the depth perception, another visual and spatial deficit, or combinations thereof of the patient. The neural target site can be the subthalamic nucleus, the globus pallidum, the putamen, the nucleus accumbens, the ventral the striatum, the ventral internal capsule, a thalamic relay nucleus, or combinations thereof. The method can also include asking the patient to perform a task reliant on depth perception prior to delivering the focused ultrasound signal.

### BRIEF DESCRIPTION OF THE DRAWINGS

FIG. 1 illustrates a method for providing treatment via focused ultrasound treatment;

FIG. 2 represents a method for screening a patient for a specific disorder;

FIG. 3 illustrates a method for using cues to generate features used to screen patients for a disorder;

FIG. 4 illustrates a method for extracting connectivity features for evaluating a patient for focused ultrasound treatment;

FIG. 5 illustrates a method for generating features from imaging for extracting connectivity features for evaluating a patient for focused ultrasound treatment;

FIG. 6 illustrates one method for monitoring a patient to predict or detect an onset of symptoms associated with a disorder for a patient;

FIG. 7 illustrates another example of a method for evaluating a patient for an onset of symptoms requiring treatment via focused ultrasound treatment;

FIG. 8 illustrates a method for targeting focused ultrasound treatment in a brain of a patient for treatment of a disorder;

FIG. 9 illustrates a another method for targeting focused ultrasound treatment in a brain of a patient for treatment of a disorder using acute feedback;

FIG. 10 illustrates a method for selecting a dose for focused ultrasound treatment;

FIG. 11 illustrates a system for providing treatment to a patient via focused ultrasound treatment;

FIG. 12 illustrates a system for targeting focused ultrasound treatment for treatment or diagnosis of a disorder to a specific region of the brain of a patient;

FIG. 13 illustrates a system for determining a presence or risk of a disorder from imaging of a brain of a patient;

FIG. 14 illustrates one example of a system for diagnosis and monitoring of disorders; and

FIG. 15 is a schematic block diagram illustrating an exemplary system of hardware components.

### DETAILED DESCRIPTION

The term “wellness” as used herein in intended to refer to the mental, physical, cognitive, behavioral, social, and emotional health of a user and should be construed to cover each of the health, function, balance, resilience, homeostasis, disease, and condition of the user. In various examples herein, the wellness of the user can be related to the readiness of the user to perform job-related, athletic, or everyday functions, enter into a flow or zone state, the susceptibility of the user to an infectious disease, the ability of the user to resist the effects of addiction, worsening pain of a user, increased or reduces stress, anxiety, a quality of life of the user, and similar qualities of a user.

A “wellness-relevant parameter” is a parameter that is relevant to the wellness of a user.

A “biological rhythm” is any chronobiological phenomenon that affects human beings, including but not limited to, circadian rhythms, ultradian rhythms, infradian rhythms, diurnal cycle, sleep/wake cycles, and patterns of life.

A “focused ultrasound treatment” as described herein, is any treatment that applies ultrasound to the brain for the purpose of neuromodulation and modulating neural activity or disrupting the blood-brain barrier for the purpose of allowing targeted entry of therapeutics into the brain. Accordingly, a focused ultrasound treatment can involve the use of ultrasound to modulate neural activity, the use of ultrasound in combination with microbubbles to disrupt the blood-brain barrier and allow selective entry of an appropriate therapeutic into the brain, or a combination of blood-brain barrier disruption with neuromodulation of appropriate targets. It will be appreciated that the use of focused ultrasound for disrupting the blood-brain barrier will involve a degree of neuromodulation due to the impact of the ultrasound on the surrounding tissue, but references to a focused ultrasound treatment comprising a combination of neuromodulation and disruption of the blood-brain barrier refers to at least two separate applications of focused ultrasound, with a first application of focused ultrasound targeted at a location at which penetration of therapeutics into the brain is desired and a second application of focused ultrasound at a location with the brain tissue for which neuromodulation is desired.

A “portable monitoring device,” as used herein, refers to a device that is worn by, carried by, or implanted within a user that incorporates either or both of an input device and user interface for receiving input from the user and sensors for monitoring either a wellness-relevant parameter or a parameter that can be used to calculate or estimate a wellness-relevant parameter.

An “index”, as used herein, is intended to cover composite statistics derived from a series of observations and used as an indicator or measure. An index can be an ordinal, continuous, or categorical value representing the observations and correlations, and should be read to encompass statistics traditionally referred to as “scores” as well as the more technical meaning of index.

A “clinical parameter”, as used herein, can be any continuous or categorical parameter representing the mental, physical, cognitive, behavioral, social, and emotional health of a user and can represent any or all of the health, function, a zone or flow state, balance, resilience, homeostasis, disease, and condition of the user.

A “continuous parameter,” as used herein, is used broadly to refer to a parameter that can assume any value within a predefined range to a predetermined level of precision. Accordingly, a value is referred to herein as a continuous parameter even if limited, in practice, to a finite number of discrete values within the range by the resolution at which the value is measured, calculated, or stored.

An “physiological sensing device,” as used herein, is a device to measure one or more physiological parameters and/or biological rhythms. A physiological sensing device is often implanted, ingested, or wearable, although in some instances an off-body device can be used to capture physiological parameters.

A “portable computing device,” as used herein, is a computing device that can carried by the user, such as a smartphone, smart watch, tablet, notebook, and laptop, that can measure a wellness-relevant parameter either through sensors on the device or via interaction with the user. A portable computing device can include, for example, a user interface for receiving an input from the user, kinematic sensors for measuring activity by the user, and location services that track a location of the user.

As used herein, a “predictive model” is a mathematical model or machine learning model that either predicts a future state of a parameter or estimates a current state of a parameter that cannot be directly measured.

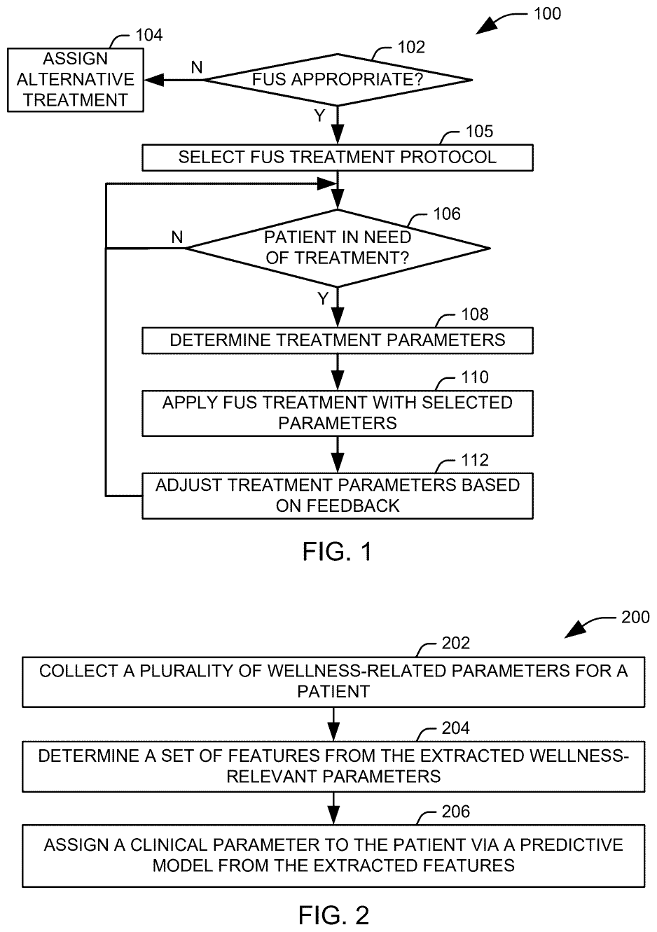

FIG. 1 illustrates a method 100 for providing treatment via focused ultrasound treatment. At 102, a potential patient is screened to determine if focused ultrasound (FUS) treatment is appropriate for that patient. In one implementation, the screening is performed by collecting a plurality of wellness-relevant parameters for the patient that have been determined to be relevant to a particular disorder and providing them to a predictive model to assign a clinical parameter to the patient associated with the likelihood that the patient would benefit from focused ultrasound treatment.

FIG. 2 represents a method 200 for screening a patient for a specific disorder. At 202, a plurality of wellness-related parameters are collected for the patient. Parameters can be retrieved from an electronic health records (EHR) interface and/or other available databases, including, for example, employment information (e.g., title, department, shift), age, sex, home zip code, genomic data, nutritional information, medication intake, household information (e.g., type of home, number and age of residents), social and psychosocial data, consumer spending and profiles, financial data, food safety information, the presence or absence of physical abuse, and relevant medical history. Parameters can also be measured via diagnostic systems, imaging systems, wearable sensors, cognitive tests, questionnaires, and other means. As noted above, wellness-relevant parameters can include at least physiological, cognitive, motor/musculoskeletal, sensory, sleep, biomarkers and behavioral parameters. Table I provides non-limiting examples of physiological parameters that can be measured and exemplary tests, devices, and methods, to measure the physiological parameters.

TABLE I

| Physiological | Exemplary Devices and Methods to Measure |
|---|---|
| Parameter | Physiological Parameters |
| Brain Activity | Electroencephalogram, Magnetic Resonance |
| | Imaging, including functional Magnetic Resonance |
| | Imaging (fMRI), PET, SPECT, MEG, near-infrared |
| | spectroscopy, functional near-infrared spectroscopy, |
| | and other brain imaging modalities looking at |
| | electrical, blood flow, neurotransmitter, and |
| | metabolic MRI, taken either during a cognitive task |
| | or while the patient is at rest |
| Brain Structure | Magnetic Resonance Imaging |
| Heart rate | Electrocardiogram and Photoplethysmogram |
| Heart rate | Electrocardiogram, Photoplethysmogram |
| variability | |
| Eye tracking | Pupillometry, including tracking saccades, fixations, |
| | and pupil size (e.g., dilation) |
| Perspiration | Perspiration sensor |
| Retinal | OCT and angiography |
| Retinal anatomy, | Other Retinal imaging including wide field |
| layers, retinal | |
| vasculature | |
| Blood pressure | Sphygmomanometer |
| Body temperature | Thermometer, infrared thermography |
| Blood oxygen | Pulse oximeter/accelerometer |
| saturation and | |
| respiratory rate | |
| Skin conductivity | Electrodermal activity |
| Facial emotions | Camera or EMG based sensors for emotion and |
| | wellness |
| Sympathetic and | Derived from the above measurements |
| parasympathetic | |
| tone | |

The physiological parameters can be measured in clinical settings with appropriate devices or in non-clinical settings via wearable, implantable, or portable devices. Some information can also be determined from self-reporting by the user via applications in a mobile device or via interaction with applications on the mobile device. For example, a smart watch, ring, or patch can be used to measure the user's heart rate, heart rate variability, body temperature, blood oxygen saturation, movement, and sleep. In a non-clinical setting, these values can also be subject to a diurnal analysis to estimate variability. Eye tracking can be performed, for example, using a camera on a mobile device and specialized software.

Table II provides non-limiting examples of cognitive parameters that are gamified and that can be measured and exemplary methods and tests/tasks to measure such cognitive parameters. The cognitive parameters can be assessed by a battery of cognitive tests that measure, for example, executive function, decision making, working memory, attention, and fatigue.

TABLE II

| | Exemplary Tests and Methods to |
|---|---|
| Cognitive Parameter | Measure Cognitive Parameters |
| Temporal discounting | Kirby Delay Discounting Task |
| Alertness and fatigue | Psychomotor Vigilance Task |
| Focused attention and | Erikson Flanker Task |
| response inhibition | |
| Working memory | N-Back Task |
| Attentional bias towards | Dot-Probe Task |
| emotional cues | |
| Inflexible persistence | Wisconsin Card Sorting Task |
| Decision making | Iowa Gambling Task |
| Risk taking behavior | Balloon Analogue Risk Task |
| Inhibitory control | Anti-Saccade Task |
| Sustained attention | Sustained Attention |
| Executive function | Task Shifting or Set Shifting Task |
| Long term memory | Identifying pictures of famous people |
| | and other memory related tasks |

These cognitive tests can be administered in a clinical/laboratory setting or in a naturalistic, non-clinical setting such as when the user is at home, work or other non-clinical setting. A smart device, such as a smartphone, tablet, or smart watch, can facilitate measuring these cognitive parameters in a naturalistic, non-clinical setting. For example, the Erikson Flanker, N-Back and Psychomotor Vigilance Tasks can be taken via an application on a smart phone, tablet, or smart watch. In one example, the patient can be allowed to explore a virtual reality environment and collect items within the environment. The patient is then asked to recount where each item was found within the virtual environment and the relationship of that location to a starting point, testing the patient's ability to recall spatial relationships among the virtual locations.

TABLE III provides non-limiting examples of parameters associated with movement and activity of the user, referred to herein alternatively for ease of reference as “motor parameters,” that can be measured and exemplary tests, devices, and methods. The use of portable monitoring, physiological sensing, and portable computing devices allows the motor parameters to be measured. Using embedded accelerometer, GPS, and cameras, the user's movements can be captured and quantified to see how wellness affects them and related to the wellness-relevant parameters. Range of motion and gait analysis can be performed in a clinical setting using appropriate motion capture and camera equipment for evaluation.

TABLE III

| Motor/Musculoskeletal | Exemplary Tests and Methods to Measure |
|---|---|
| Parameter | Motor/Musculoskeletal Parameters |
| Activity level | Daily movement total, time of activities, from |
| | wearable accelerometer, steps, Motion Capture |
| | data, gait analysis, GPS, deviation from |
| | established geolocation patterns, force plates |
| Gait analysis | Gait mat, camera, force plats |
| Range of motion | Motion capture, camera, |

TABLE IV provides non-limiting examples of parameters associated with sensory acuity of the user, referred to herein alternatively for ease of reference as “sensory parameters,” that can be measured and exemplary tests, devices, and methods.

TABLE IV

| | Exemplary Tests and Methods to Measure |
|---|---|
| Sensory Parameter | sensory Parameters |
| Vision | Visual acuity test, visual field tests, eye tracking, |
| | EMG |
| Hearing | Hearing tests |
| Touch | Two-point discrimination, frey filament |
| Smell/taste | |
| Vestibular | Vestibula function test |

TABLE V provides non-limiting examples of parameters associated with a sleep quantity, phases, and quality of the user, referred to herein alternatively for ease of reference as “sleep parameters,” that can be measured and exemplary tests, devices, and methods.

TABLE V

| | Exemplary Tests and Methods to Measure Sleep |
|---|---|
| Sleep Parameter | Parameters |
| Sleep from | Sleep onset & offset, sleep quality, sleep quantity, |
| wearables | from wearable accelerometer, temperature, and |
| | PPG, |
| Sleep Questions | Pittsburg Sleep Quality Index, Functional Outcomes |
| | of Sleep Questionnaire, Fatigue Severity Scale, |
| | Epworth Sleepiness Scale |
| Devices | Polysomnography; ultrasound, camera, bed |
| | sensors, EEG |
| Circadian Rhythm | Light sensors, actigraphy, serum levels, core body |
| | temperature |

TABLE VI provides non-limiting examples of parameters extracted by locating biomarkers associates with the user, referred to herein alternatively for ease of reference as “biomarker parameters,” that can be measured and exemplary tests, devices, and methods. Biomarkers can also include imaging and physiological biomarkers related to a state of chronic wellness and improvement or worsening of the chronic wellness state.

TABLE VI

| | Exemplary Tests and Methods to Measure |
|---|---|
| Biomarkers Parameter | Biomarkers Parameters |
| Genetic biomarkers | Genetic testing |
| Immune biomarkers | Blood, saliva, and/or urine tests |
| including TNF-alpha, | |
| immune alteration (e.g., | |
| ILs), oxidative stress, and | |
| hormones (e.g., cortisol) | |

Table VII provides non-limiting examples of psychosocial and behavioral parameters, referred to herein alternatively for ease of reference as “psychosocial parameters,” that can be measured and exemplary tests, devices, and methods.

TABLE VII

| Psychosocial or | Exemplary Tests and Methods to Measure |
|---|---|
| Behavioral Parameter | Psychosocial or Behavioral Parameters |
| Symptom log | Presence of specific symptoms (i.e., fever, |
| | headache, cough, loss of smell) |
| Medical Records | Medical history, prescriptions, setting for |
| | treatment devices such as spinal cord |
| | stimulator, imaging data |
| Wellness Rating | Visual Analog Scale, Defense & Veterans |
| | wellness rating scale, wellness scale, |
| | Wellness Assessment screening tool and |
| | outcomes registry |
| Burnout | Burnout inventory or similar |
| Physical, Mental, and | User-Reported Outcomes Measurement |
| Social Health | Information System (PROMIS), Quality of |
| | Life Questionnaire |
| Depression | Hamilton Depression Rating Scale |
| Anxiety | Hamilton Anxiety Rating Scale |
| Mania | Snaith-Hamilton Pleasure Scale |
| Mood/ | Profile of Mood States; Positive Affect |
| Catastrophizing scale | Negative Affect Schedule |
| Affect | Positive Affect Negative Affect Schedule |
| Impulsivity | Barratt Impulsiveness Scale |
| Adverse Childhood | Childhood trauma |
| Experiences | |
| Daily Activities | Exposure, risk taking |
| Daily Workload and | NASA Task Load Index, Perceived Stress Scale |
| Stress | (PSS), |
| | Social Readjustment Rating Scale (SRRS) |
| Social Determents of | Social determents of health questionnaire |
| Health | |

The behavioral and psychosocial parameters can measure the user's functionality as well as subjective/self-reporting questionnaires. The subjective/self-reporting questionnaires can be collected in a clinical/laboratory setting or in a naturalistic, in the wild, non-clinical setting such as when the user is at home, work, or other non-clinical setting. A smart device, such as a smartphone, tablet, or personal computer can be used to administer the subjective/self-reporting questionnaires. Using embedded accelerometers and cameras, these smart devices can also be used to capture the facial expression analysis to analyze the user's facial expressions that could indicate mood, anxiety, depression, agitation, and fatigue. This affect detection can be performed using an appropriate predictive model trained on faces of users mimicking or experiencing a given emotion or mental state.

Additional elements of monitoring can include the monitoring of the user's compliance with the use of a smart phone, TV, portable device, a portable device. For example, a user may be sent messages by the system inquiring on their wellness level, general mood, or the status of any other wellness-relevant parameter on the portable computing device. A measure of compliance can be determined according to the percentage of these messages to which the user responds via the user interface on the portable computing device and used as an indication as to how likely the patient is to comply with treatment protocols and monitoring. Further, where parameters cannot be readily extracted from wearable or portable devices, they can be retrieved from an electronic health records (EHR) database. Biomarker and motion parameters, in particular, may be retrieved from the EHR, along with other parameters including medical history, prescribed medications, demographic parameters, age, height, weight, and other medically relevant parameters.

At 204, a set of features are determined from the extracted wellness-relevant parameters as categorical and continuous parameters representing the wellness-relevant parameters. In one example, the parameters can include descriptive statistics, such as measures of central tendency (e.g., median, mode, arithmetic mean, or geometric mean) and measures of deviation (e.g., range, interquartile range, variance, standard deviation, etc.) of time series of the monitored parameters, as well as the time series themselves. Specifically, the feature set provided to the predictive model can include, for at least one parameter, either two values representing the value for the parameter at different times or a single value, such as a measure of central tendency or a measure of deviation which represents values for the parameter across a plurality of times.

In one implementation, features can be generated via a wavelet transform on a time series of values for one or more parameters to provide a set of wavelet coefficients. It will be appreciated that the wavelet transform used herein is two-dimensional, such that the coefficients can be envisioned as a two-dimensional array across time and either frequency or scale.

For a given time series of parameters, xi, the wavelet coefficients, Wₐ(n), produced in a wavelet decomposition can be defined as:

$${W}_{a} ( n ) = {a}^{- 1} {\sum }_{i = 1}^{M} {x}_{i} ψ ( \frac{i - n}{a} ) \qquad Eq . 1$$

wherein ψ is the wavelet function, M is the length of the time series, and a and n define the coefficient computation locations.

A facial expression classifier (not shown) can evaluate recorded data from a camera and/or recorded images or videos of the user's face from a smartphone or other mobile device, to assign an emotional state to the user at various times throughout the day. The extracted features can be categorical, representing the most likely emotional state of the user, or continuous, for example, as a time series of probability values for various emotional states (e.g., anxiety, discomfort, anger, etc.) as determined by the facial expression classifier. One or more image classifiers that reduce provided medical images to categorical or continuous features for use at the predictive model. For example, the image classifiers can generate connectivity parameters representing the interconnectivity of different locations within the patient's brain, parameters representing the structure of function of the brain, parameters representing the vasculature of the brain or eye, and parameters representing eye movement and pupil dilation. It will be appreciated that each of the facial expression classifier and the one or more image classifiers can be implemented using one or more of the models discussed below for use in the predictive model.

In one implementation, the extracted features from the set of wellness-relevant parameters can be collected into a set of aggregate parameters. It will be appreciated that each aggregate parameter can be a weighted combination of the set of wellness-relevant parameters, functions of parameters from the set of wellness-relevant parameters, or features extracted from the wellness-relevant parameters. Accordingly, a given aggregate parameter can represent a plurality of wellness-relevant parameters, and, in general, the plurality of wellness-relevant parameters represented by each aggregate parameter will be related, such that the aggregate parameter represents a specific domain of wellness for the user. In general, each aggregate parameters can use parameters from various sources. In some implementations, the aggregate parameters can be provided to multiple predictive models (not shown) that each receive a unique proper subset of the aggregate parameters. Each predictive model can provide a different clinical parameter representing a different aspect of the user's wellness, such that the aggregate parameters can be utilized for multiple purposes in evaluating the wellness of the user.

It will be appreciated that the specific parameters and features used for screening can vary with the implementation. For example, screening for pain may focus on measured autonomic parameters, sleep parameters, data from the patient's medical record, functional brain imaging, and self-reporting from the patient. In another example, screening for addiction, PTSD, phobias, panic disorders, anxiety, depression, and schizophrenia may focus on autonomic, behavioral, cognitive and psychosocial parameters, data from the patient's medical records, and self-reporting from the patient on various questionnaires. Screening for dementia may focus on brain and eye imaging, autonomic, psychosocial, sensory, biomarker, and cognitive parameters, and data from the patient's medical record. The patient's response, both self-reported and in the form of measured physiological values, to cues appropriate to their disorder can also be recorded and used for generating features, particularly for addiction, depression, PTSD, anxiety, and phobias. Screening for movement disorders such as Parkinson's disease (PD) and Huntington's disease, or for the aftereffects of strokes can focus on motor, autonomic, psychosocial, and cognitive parameters. It will be appreciated that the methods herein focus on monitoring and improving the behavioral and cognitive and autonomic nervous system aspects of these diseases with our approach. As a specific example, our approach helping to improve visuospatial impairments in Parkinson's disease such as visual perception, blurry vision, judging distances, depth perception, and other visual and spatial deficits that impact so many PD patients. Screening for obsessive compulsive disorder and neurodevelopment disorders, such as autism and autism related disorders, can focus on autonomic, psychosocial, and cognitive parameters.

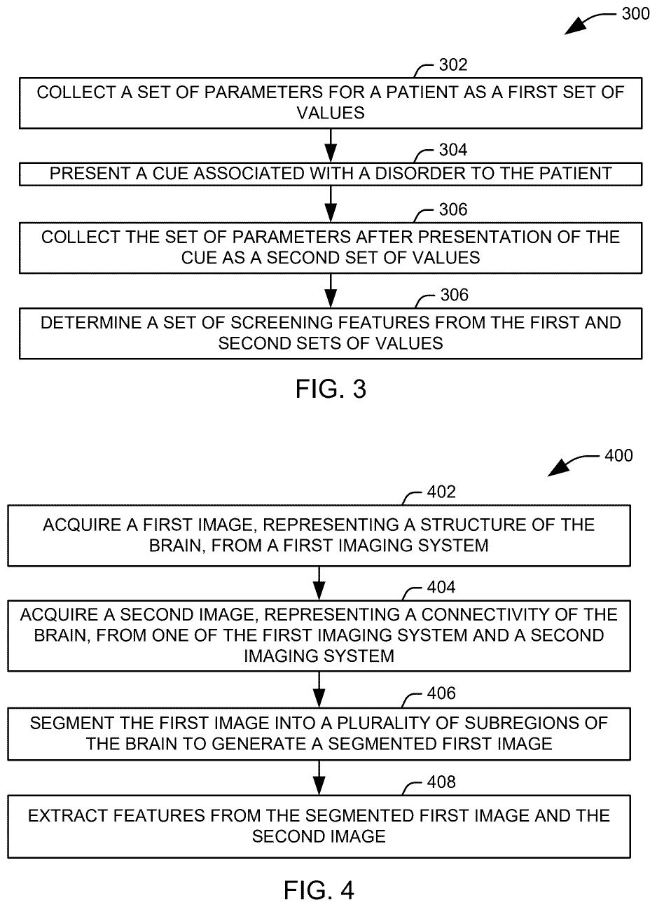

FIG. 3 illustrates a method 300 for using cues to generate features used to screen patients for a disorder. It will be appreciated that the method 300 describes eliciting responses to cues in the context of patient screening, that cue-induced responses can also be used in detecting or predicting the onset of symptoms for a patient, for determining, refining, and optimizing an appropriate target location or appropriate parameters for focused ultrasound treatment, or for evaluating the effectiveness of focused ultrasound treatment. The presentation of cues can also be used as part of treatment for various disorders, as will be discussed in more detail below. At 302, a set of parameters representing the disorder is collected from the patient as a first set of values. The specific parameters will vary with the disorder for which the patient is being screened, but in general, the parameters can include the patient's response to various standard questionnaires as well as physiological parameters measured from the patients. At 304, a cue associated with the disorder is presented to the patient. It will be appreciated that the cue will be selected to be specific to the disorder, and can include not only visual cues but auditory, gustatory, tactile, and olfactory cues. For example, if the patient suffers from addiction to alcohol, the cue can be selected to represent the sound of a beer bottle opening, or the taste or smell of the patient's preferred alcoholic beverage. It will be appreciated that cues may be simulated using virtual or augmented reality systems. One example of the presentation of cues can be found in U.S. Patent Publication No. US 2021/0162217, filed Dec. 2, 2020, and entitled “METHODS AND SYSTEMS OF IMPROVING AND MONITORING ADDICTION USING CUE REACTIVITY,” which is hereby incorporated by reference in its entirety. At 306, the set of parameters representing the disorder is collected from the patient after presentation of the cue as a second set of values. At 308, a set of screening features are determined from the first set of values and the second set of values. In one example, the screening features are determined as functions of the first and second values for a given parameter, such as a difference or a ratio between the values.

Additional features can be extracted from imaging taken of the patient. One set of features can represent connectivity of different regions of the brain, as differences in connectivity can be indicative of different levels of risks of various disorders. FIG. 4 illustrates a method 400 for extracting connectivity features for evaluating a patient for focused ultrasound treatment. It will be appreciated that the method 400 describes extracting connectivity features in the context of patient screening, that connectivity features can also be used in detecting or predicting the onset of symptoms for a patient, for determining an appropriate location or appropriate parameters for focused ultrasound treatment, or for evaluating the effectiveness of focused ultrasound treatment. At 402 a first image, representing a structure of the brain, is acquired from a first imaging system. At 404, a second image, representing a connectivity of the brain, is acquired from one of the first imaging system and a second imaging system. In one implementation, the second image is acquired via diffusion tensor imaging. At 406, the first image is segmented into a plurality of subregions of the brain to generate a segmented first image, such that each of at least a subset of a plurality of voxels comprising the first image are associated with one of the plurality of subregions. It will be appreciated that not all portions of the first image may be of interest for a given disorder, and thus only those voxels representing the plurality of subregions may be included in the segmentation. At 408, features are extracted from the segmented first image and the second image. In one example, the image is provided directly to a deep learning system, such as a convolutional neural network, such that the individual pixel intensity or chromaticity values in the image are the extracted features. Alternatively, other connectivity parameters may be specific to the disorder, representing the density of connections between a first region of interest associated with a possible focused ultrasound treatment target and other regions of interest within the brain.

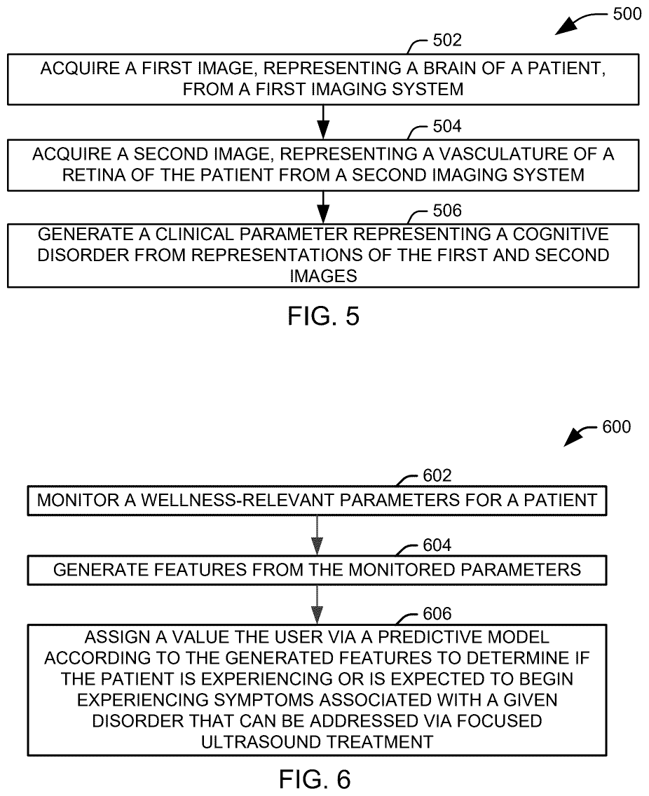

In another implementation, FIG. 5 illustrates a method 500 for generating features from imaging of the eye for evaluating a patient for focused ultrasound treatment. It will be appreciated that the method 500 describes extracting parameters from imaging of the eye in the context of patient screening, that features representing the retina, the optic nerve, and associated vasculature of these structures can also be used in detecting or predicting the onset of symptoms for a patient, for determining an appropriate location or appropriate parameters for focused ultrasound treatment, or for evaluating the effectiveness of focused ultrasound treatment. At 502, a first image, representing a brain of a patient, is acquired from a first imaging system. In one example, the first imaging system is a magnetic resonance imaging (MRI) imager. Additionally or alternatively, the first image can be acquired via diffusion tensor imaging. In one example, the representation of the first image represents a cortical profile of the brain. In another example, the representation of the first image represents a vasculature of the brain. In a further example, the representation of the first image represents protein plaques/tangles within the brain, such as beta-amyloid or tau proteins. In a still further example, the representation of the first image represents a connectivity of the brain. At 504, a second image, representing one of a retina, optic nerve, and associated vasculature of the patient, is acquired from a second imaging system. In one example, the second image is an optical coherence tomography angiography image.

At 506, features are extracted from each of the first image and the second image for use in a predictive model. In one example, it will be appreciated that the representation of each image can include the chromaticity values associated with each pixel or voxel in the image, such that all or most of the data associated with each image is provided to the machine learning system. Alternatively, a set of numerical features representing the content of each image can be extracted, such as total areas or volumes of structures or tissues of interest. In one example, a pupil of the patient can be imaged to provide an additional parameter representing at least one of eye tracking data, eye movement, pupil size, and a change in pupil size. In one example, various biometric parameters extracted from the brain images include parameters related to connectivity, gyrification, volume, vascular load, networks, a white matter load, a grey/white mater ratio, cortical thickness, sulcal depth and other measurements. Images of the brain can also be segmented to determine these parameters for specific regions of the brain. Similarly, numerical parameters can be extracted from the images of the eye, including a volume, thickness, or texture of the retina or individual retinal layers at one or more locations or an average thickness of the retina or one or more layers across locations, values representing the vascular pattern and density of the retina or individual layers of the retina, a size of the foveal avascular zone, a height, width, or volume of the optic chiasm, a height, width, or volume of the intraorbital optic nerve, a height, width, or volume of the intracranial optic nerve, a total area of the vasculature in the image.

Returning to FIG. 2, at 206, a clinical parameter is assigned to the patient via a predictive model from the extracted features. The predictive model can utilize one or more pattern recognition algorithms, each of which analyze the extracted features or a subset of the extracted features to assign a continuous or categorical clinical parameter to the user representing the likelihood that the patient would benefit from focused ultrasound treatment. In one example, the clinical parameter can be a continuous parameter representing the likelihood that the patient has a disorder that can be treated via focused ultrasound treatment, the likelihood that the patient has a specific disorder, such as Alzheimer's disease, that can be treated via focused ultrasound treatment, the likelihood that a patient will benefit from focused ultrasound treatment generally given a known diagnosis, or the likelihood that the patient will benefit from focused ultrasound treatment in a specific location or region of the brain. In another example, the clinical parameter can be a categorical parameter representing whether the patient has a disorder that can be treated via focused ultrasound treatment, categories representing changes in symptoms associated with a disease or disorder (e.g., “improving”, “stable, “worsening”), categories representing a predicted response to focused ultrasound treatment generally or at a specific location or region of the brain, whether the patient has a specific disorder, such as Alzheimer's disease, that can be treated via focused ultrasound treatment, the severity of a disorder, whether a patient will benefit from focused ultrasound treatment given a known diagnosis, or categories representing ranges of likelihoods that the patient falls into one of these categories.

Where multiple classification or regression models are used, an arbitration element can be utilized to provide a coherent result from the plurality of models. The training process of a given classifier will vary with its implementation, but training generally involves a statistical aggregation of training data into one or more parameters associated with the output class. The training process can be accomplished on a remote system and/or on the local device or wearable, app. The training process can be achieved in a federated or non-federated fashion. For rule-based models, such as decision trees, domain knowledge, for example, as provided by one or more human experts or extracted from existing research data, can be used in place of or to supplement training data in selecting rules for classifying a user using the extracted features. Any of a variety of techniques can be utilized for the classification algorithm, including support vector machines, regression models, self-organized maps, fuzzy logic systems, data fusion processes, boosting and bagging methods, rule-based systems, or artificial neural networks.

Federated learning (aka collaborative learning) is a predictive technique that trains an algorithm across multiple decentralized edge devices or servers holding local data samples, without exchanging their data samples. This approach stands in contrast to traditional centralized predictive techniques where all data samples are uploaded to one server, as well as to more classical decentralized approaches which assume that local data samples are identically distributed. Federated learning enables multiple actors to build a common, robust predictive model without sharing data, thus addressing critical issues such as data privacy, data security, data access rights, and access to heterogeneous data. Its applications are spread over a number of industries including defense, telecommunications, IoT, or pharmaceutics.

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. In one implementation, the SVM can be implemented via a kernel method using a linear or non-linear kernel.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier. Another example is utilizing an autoencoder to detect outlier in wellness-relevant parameters as an anomaly detector to identify when various parameters are outside their normal range for an individual.

Many ANN classifiers are fully connected and feedforward. A convolutional neural network, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a modified version of recurrent neural networks, which makes it easier to remember past data in memory.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used.

In some implementations, the predictive model can be retrained to tune various parameters of the model based upon the accuracy of predictions made by the model. Parameters associated with the model, such as internal weights and thresholds for producing categorical inputs or outputs from continuous values, can be adjusted according to the differences in the actual and predicted outcomes. In one example, an actual value for the clinical parameter for a given patient can be determined as a categorical or continuous outcome, for example, based on a degree of improvement for the patent, a diagnosis, or other appropriate outcome, and combined with the set of features for the patient to provide additional training samples for the model.

Returning to FIG. 1, if it is determined that focused ultrasound treatment is not appropriate for the patient (N), the method advances to 104, where the patient is assigned to an alternative treatment. Alternative treatments can include any of medications, transcranial magnetic stimulation, deep brain stimulation, biologicals, surgical intervention, changes of settings for an existing spinal cord stimulator, behavioral and social intervention, digital intervention via a portable device, mindfulness approaches, social media approaches, a care provider coming to the individual, directing an individual to go to a clinic, emergency room, or hospital, or directing the user to obtain additional testing. If it is determined that focused ultrasound treatment is appropriate (Y), the method advances to 105, where a specific focused ultrasound treatment protocol is selected. It will be appreciated that the specific focused ultrasound treatment protocol can vary with the severity of the disorder. If the patient is determined to be at high risk for a disorder or is determined to have a mild or moderate disorders, they can be assigned neuromodulation via focused ultrasound. Patients with more severe presentations of a disorder can be assigned to a more intensive treatment, such as the application of drugs or antibodies with targeted disruption of the blood-brain barrier of the patient via focused ultrasound in combination with doses of microbubbles provided to the bloodstream of the patient, although it will be appreciated that a patient can receive focused ultrasound treatments to disrupt the blood-brain barrier in addition to neuromodulation via focused ultrasound. In one example, for treatment of neurodegeneration, the treatment can utilize a therapeutic to remove protein plaques or other pathological substances within the brain (i.e. tau tangles), such as an anti-beta amyloid antibody (e.g., Aducanumab, Lecanemab, etc) In the instance of the therapeutic agent being an anti-beta amyloid antibody (e.g., that target beta amyloid and its constituents), such an antibody (e.g. Aducanumab) can be delivered intravenously, for example, at a dose of at least 0.1 mg/kg, 1 mg/kg, 3 mg/kg, or 6 mg/kg. Other doses could be delivered as well for other antibodies. In certain aspects, the therapeutic agent is Lecanemab which can have a dose of, for example, 10 mg/kg every two weeks. Lecanemab targets a soluble, “protofil’ version of amyloid-beta and also binds, albeit more weakly, to the extracellular amyloid deposits known as plaques that Aduhelm bind to primarily. Other therapeutics can be used in combination with targeted disruption of the blood-brain barrier for other disorders.

The method then advances to 106, where the patient is monitored to predict or detect an onset of symptoms. It will be appreciated that the timing for focused ultrasound treatment can be personalized and depend on both the onset of symptoms and the progression of the disorder as well as various treatment protocols associated with focused ultrasound modality and any therapeutics introduced during treatment.

FIG. 6 illustrates one method 600 for monitoring a patient to predict or detect an onset of symptoms associated with a disorder for a patient. At 602, parameters are monitored for a patient. It will be appreciated that the monitored parameters can include any of the parameters discussed in Tables I-VII, and that the parameters can be monitored, for example, using wearable devices, portable devices, such as mobile phones, tablets, and personal computers, or from periodically querying an electronic health records system. At 604, appropriate features are generated from the monitored parameters. At least a subset of the monitored parameters can be represented by features representing a change in the parameter over time, and can be represented as a time series, or a descriptive statistic, such as a measure of central tendency or a measure of deviation which represents values for the parameter across a plurality of times.

At 606, the extracted parameters can be provided to a predictive model to determine if the patient is experiencing or is expected to begin experiencing symptoms associated with a given disorder that can be addressed via focused ultrasound treatment. For example, the predictive model can predict or detect an onset of cravings for a patient being treated for addiction, episodes of pain for patients treated for chronic pain, a worsening of impairment caused by a neurodegenerative disorder, hallucinations or delusions associated with schizophrenia, or an acute episode associated with depression, PTSD, drug craving, behavioral addiction, or anxiety disorder. In one example, the predictive model can assign a continuous parameter that corresponds to a likelihood that that user has or is about to have symptoms associated with a disorder, an increase in stress for the patient, an increased in anxiety for the patient, a likelihood that the user will experience an intensifying of symptoms associated with the disorder, a likelihood that the user will use an addictive substance or engage in an addictive behavior during rehabilitation or treatment, an increase in cravings for an addictive substance or addiction behavior, a current or predicted level of pain for the user, an expected performance level of the user associated with a current or future time for a particular activity or occupation, or a change in symptoms associated with a disease or disorder.

In another example, the predictive model can assign a categorical parameter that corresponds to ranges of the likelihoods described above, the presence or predicted presence of a specific disease or disorder, a set of categories representing the patient's readiness for a particular activity or occupation, categories representing changes in symptoms associated with a disease or disorder (e.g., “improving”, “stable, “worsening”), or categories representing a status of the user (e.g., normal,” “stressed”, “μl”). In one implementation, the predictive model can include a constituent model that predicts future values for the aggregate parameters, such as a convolutional neural network that is provided with one or more two-dimensional arrays of wavelet transform coefficients as an input. The wavelet coefficients detect changes not only in time, but also in temporal patterns, and can thus reflect changes in the ordinary biological rhythms of the user. It will be appreciated that a given constituent model can use data in addition to the aggregate parameters, such as other extracted features to provide these predictions. Additionally, or alternatively, the predictive model can use constituent models that predict current or future values for the aggregate parameters, with these measures then used as features for generating the output of the predictive model.

In one example, the predictive model is an anomaly detection model, which detects deviations from expected values within a feature space and determines when these deviations are significant. The anomaly detection model can be trained on data from the user, which establishes a baseline of expected values for the user, and/or on data collected from other users. In one example, training the predictive model is initially trained on data collected from other users while values for the subset of the set of wellness-relevant parameters are collected from the user over a period of time. Once a sufficient amount of data is available for the user, the predictive model is retrained on the collected values for the subset of the set of wellness-relevant parameters.

In one example, in which the disorder is either addiction, a phobia, anxiety disorder, or post-traumatic stress disorder, the predictive model can utilize, among other parameters, self-reported anxiety, as well as parameters associated with anxiety, such as physiological parameters and sleep parameters. In another example, in which the disorder is a neurodegenerative disorder, the predictive model can utilize, among other parameters, cognitive tests presented to the patient in either a clinical or non-clinical environment, for example, via a smartphone, tablet, or home computer. Additionally, sleep parameters and imaging biomarkers can be used to track the progression of the disease and the need for additional treatments. In a further example, in which the disorder is depression, chronic pain, or schizophrenia, self-reporting of the patient via questionnaires as well as physiological data gathered from wearable and portable devices. Sleep data can also be utilized for monitoring depression and chronic pain.

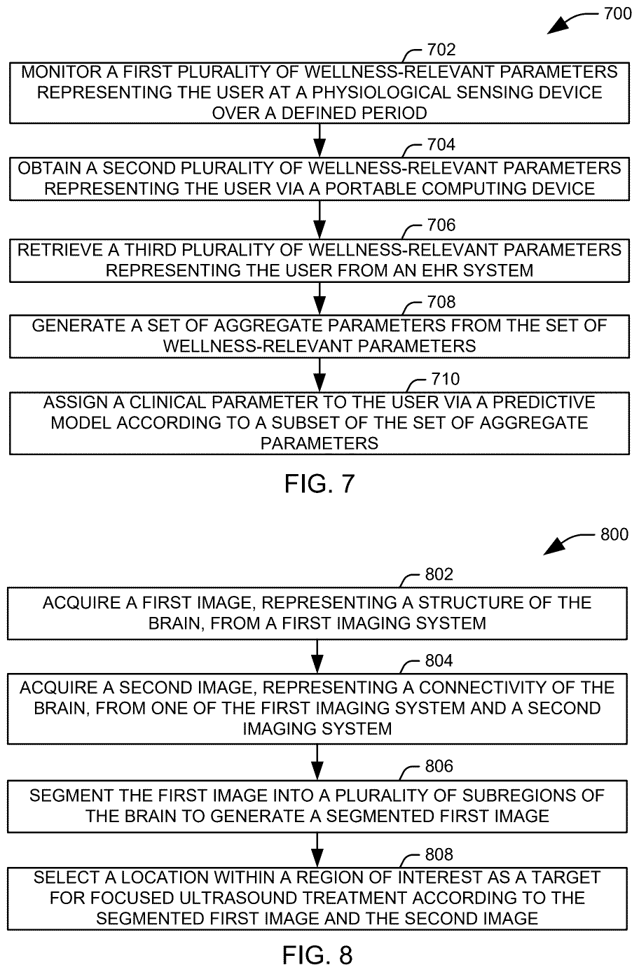

FIG. 7 illustrates another example of a method 700 for evaluating a patient for an onset of symptoms requiring treatment via focused ultrasound treatment. The result of the method is a clinical parameter representing whether the patient is experiencing or is expected to experience symptoms that would require focused ultrasound treatment. At 702, a first plurality of wellness-relevant parameters representing the user is monitored at a physiological sensing device over a defined period. In one example, the first plurality of wellness-relevant parameters can include parameters representing the autonomic function of the user and parameters representing the sleep and circadian rhythms of the user. At 704, a second plurality of wellness-relevant parameters representing the user is obtained via a portable computing device. In one example, the second plurality of wellness-relevant parameters can include parameters representing the cognitive and/or sociobehavioral wellness of the user. At 706, a third plurality of wellness-relevant parameters representing the user is retrieved from an electronic health records (EHR) system. The third plurality of wellness-relevant parameters can include, for example, parameters representing the musculoskeletal health, genomics, and various biomarkers of the user. The first plurality of wellness-relevant parameters, the second plurality of wellness-relevant parameters, and the third plurality of wellness-relevant parameters collectively form a set of wellness-relevant parameters.

At 708, a set of aggregate parameters are generated from the set of wellness-relevant parameters, with each of the set of aggregate parameters comprising a unique proper subset of the set of wellness-relevant parameters. In one example, the set of aggregate parameters includes at least a first aggregate parameter representing autonomic function of the user, a second aggregate parameter representing a cognitive function of the user, and a third aggregate parameter representing a motor and musculoskeletal health of the user. In another example, the set of aggregate parameters includes at least a first aggregate parameter representing sleep and circadian rhythms of the user, a second aggregate parameter representing a sociobehavioral function of the user, and a third aggregate parameter representing a biomarkers and genomics of the user.

At 710, a clinical parameter is assigned to the user via a predictive model according to a subset of the set of aggregate parameters. In one example, the clinical parameter is a value representing an overall wellness of the user, and the subset of the set of aggregate parameters comprises the entire set of aggregate parameters. In another example, the subset of the set of aggregate parameters is a proper subset. It will be appreciated that the aggregate parameters can be provided to multiple predictive models, with each predictive model receiving a unique subset of the set of aggregate parameters. In one example, the predictive model is an anomaly detection model, which detects deviations from expected values within a feature space and determines when these deviations are significant. The anomaly detection model can be trained on data from the user, which establishes a baseline of expected values for the user, or on data collected from other users. In one example, training the predictive model is initially trained on data collected from other users while values for the subset of the set of wellness-relevant parameters are collected from the user over a period of time. Once a sufficient amount of data is available for the user, the predictive model is retrained on the collected values for the subset of the set of wellness-relevant parameters.

In one example, a wavelet decomposition is performed on the time series for at least one aggregate parameter to provide a set of wavelet coefficients, and the set of wavelet coefficients or one or more values derived from the set of wavelet coefficients can be provided to the predictive model. Additionally or alternatively, the user can be assigned a predicted value representing a future value of a given aggregate parameter according to the values for the subset of aggregate parameters, and the value assigned to the user can be assigned based on the predicted value.

Additionally or alternatively, feedback, in the form of a self-reported level of symptoms, notes from a later clinical visit, or a measured future value for a parameter, can be used to refine the predictive model. For example, the self-reported or measured value can be compared to the value assigned to the user via a predictive model, and a parameter associated with the predictive model can be changed according to the comparison. In one example, this can be accomplished by generating a reward for a reinforcement learning process based on a similarity of the measured outcome to the value assigned to the user and changing the parameter via the reinforcement learning process.

It will be appreciated that each of the wellness values and the value assigned to the user can be provided, for example, via a user interface or network interface, to one or more of the user, the user's health care provider, the user's care team, a research team, a user's workplace, a user's sports team, an insurer, or other interested entities. This allows the value to be used to make decisions about the user's care and activities. Feedback provided to the user can be used to improve the user's awareness, perception and interpretation of being in an overall positive and negative states, allowing the user to learn strategies for avoiding negative states and inducing positive states. The provided wellness data can also be used for improvement or optimization of cognitive, motor, sensory, and behavioral function as well as generally attempting to improve the user's quality of life through suggesting actions for the user in response to changes in the clinical parameter. For example, a message can be transmitted to the user's portable computing device suggesting a course of action for the user when the clinical parameter is outside of a predetermined range of values.

Returning to FIG. 1, if it is determined that the patient is not experiencing symptoms that should be addressed via focused ultrasound treatment (N), the method remains at 106. If it is determined that focused ultrasound treatment is advisable, the method advances to 108, where appropriate parameters for focused ultrasound treatment are determined. It will be appreciated that the appropriate parameters for focused ultrasound treatment can include a target location for focused ultrasound treatment as well as parameters relating to the dosage of the focused ultrasound treatment, such as a total duration of modulation, a duration of each application, a number of applications, and an intensity, or power, of each application. Example parameters for a focused ultrasound treatment can include a sonification dose, a power (<150 W), a sonication duration (<60 min), a frequency direction, a repetition time (e.g., five seconds on and ten seconds off, a pulse duration (e.g., 100 ms on, 900 ms off, continuous or burst, energy per minute (e.g. 0 J/min-290 J/min), a frequency (e.g., 0.1-3 MHz), a number of elements (e.g., 1-1024), and a waveshape form.

FIG. 8 illustrates a method 800 for targeting focused ultrasound treatment in a brain of a patient for treatment of a disorder. At 802 a first image, representing a structure of the brain, is acquired from a first imaging system. At 804, a second image, representing a connectivity of the brain, is acquired from one of the first imaging system and a second imaging system. In one implementation, the second image is one of a set of images representing the connectivity of the brain, each acquired by applying focused ultrasound treatment at a position within the region of interest associated with the image and measuring a level of activity in at least one of the plurality of subregions of the brain in response to applied focused ultrasound treatment. Additionally or alternatively, the second image can be acquired via diffusion tensor imaging.

At 806, the first image is segmented into a plurality of subregions of the brain to generate a segmented first image, such that each of at least a subset of a plurality of voxels comprising the first image are associated with one of the plurality of subregions. It will be appreciated that not all portions of the first image may be of interest for a given disorder, and thus only those voxels representing the plurality of subregions may be included in the segmentation. At 808, a location within a region of interest of the brain is selected as a target for focused ultrasound treatment according to the segmented first image and the second image. It will be appreciated that the target can be selected not only for proximity and connections to regions of the brain for which modulation is expected to be beneficial, but also to avoid connections to regions of the brain likely to result in undesirable effects. For example, accidental stimulation of the amygdala can result in anxiety or fear, which can be counterproductive in the treatment of many disorders. Small variations in the location of modulation can drastically change the amount of incidental modulation applied to undesirable regions as well as the amount of modulation applied to a desired region of the brain.

For treatment of neurodegenerative disorders via the use of focused ultrasound to open the blood-brain barrier, the specific target can be determined from imaging, specifically by locating regions having high concentrations of protein plaque or other pathological substances. Treatment for neurocognitive disorders generally can focus on the hippocampus, the entorhinal cortex, the frontal lobe, the parietal lobe, the cingulate, any cortical regions with protein plaques or other pathological substances within the brain, the nucleus basalis, the entorhinal cortex, the thalamic nuclei, the nucleus accumbens, the fornix, the mammillary bodies, the central internal capsule, and mammillothalamic tract. For treatment of addiction or anxiety, the target can be within one of the nucleus accumbens, the insula, the cingulate cortex, the dorsolateral prefrontal cortex, the intralaminar nuclei of the thalamus, and the anterior subthalamic nucleus. For treatment of the cognitive and behavioral aspects of Parkinson's disease, in particular the visual and spatial defects often associated with the disorder, the selected target can be within the subthalamic nucleus, the globus pallidum, the putamen, the nucleus accumbens, the ventral striatum, the globus pallidum, the ventral internal capsule, and the thalamic relay nuclei. For treatment of the cognitive and behavior aspects of Huntington's disease, the target can be within the caudate, the putamen, the nucleus accumbens, the ventral striatum, the globus pallidum, the ventral internal capsule, and the thalamic nuclei.

For treatment of post-traumatic stress disorder, treatment targets can be selected within any of the amygdala, the prefrontal cortex, the hippocampus, the basolateral complex of the amygdala, the lateral nucleus, the basal nucleus, the thalamus sensory nuclei, the ventromedial prefrontal cortex, the basolateral complex, the infralimbic subregion of the medial prefrontal cortex, and the nucleus accumbens. For treatment of chronic pain, treatment targets can be selected within the sensory thalamus lateral, the internal laminar nuclei of the thalamus, the sensory nucleus, the internal capsule, the periaqueductal/periventricular gray matter, the anterior cingulate cortex, the spinal cord, the vagus nerve, the insula, and the cingulate cortex. For treatment of schizophrenia, targets can be selected within the nucleus accumbens, the intra laminar nuclei, the thalamic nuclei, the olfactory, sensory, visual, gustatory cortex and corresponding thalamic relay nuclei, the medial and lateral geniculate, the relay of all tracts, the calcarine cortex, and Herschel's gyrus. For obsessive-compulsive disorder, appropriate targets can be found in the nucleus accumbens, the ventral striatum, and the ventral internal capsule. Appropriate targets for treating the cognitive and behavioral aspects of neurodevelopmental disorders such as autism and autism related disorders, can include the nucleus accumbens, the ventral striatum, the ventral internal capsule, and the thalamic nuclei. For assisting with stroke recovery, the specific target will depend on the regions damaged by the stroke.

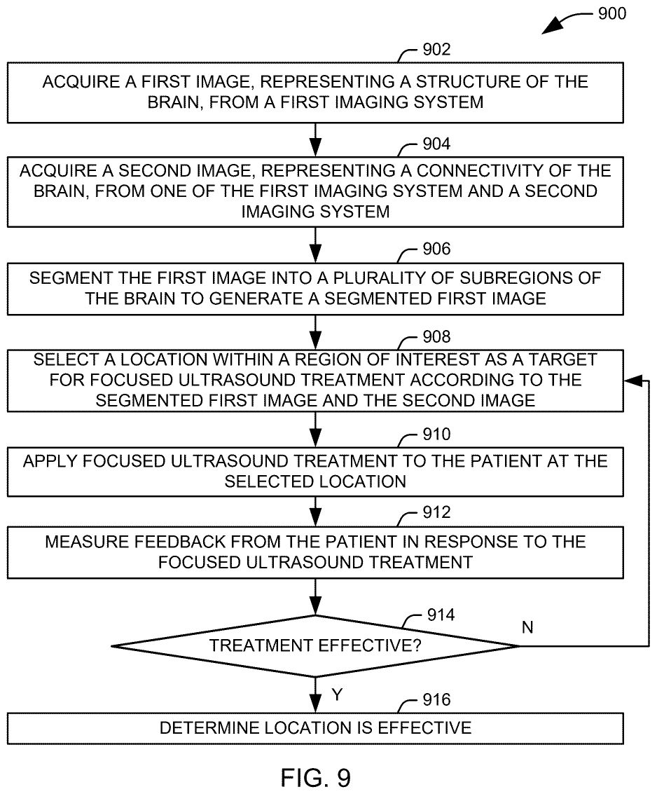

FIG. 9 illustrates another method 900 for targeting focused ultrasound treatment in a brain of a patient for treatment of a disorder using acute feedback. At 902 a first image, representing a structure of the brain, is acquired from a first imaging system. At 904, a second image, representing a connectivity of the brain, is acquired from one of the first imaging system and a second imaging system. In one implementation, the second image is one of a set of images representing the connectivity of the brain, each acquired by applying focused ultrasound treatment at a position within the region of interest associated with the image and measuring a level of activity in at least one of the plurality of subregions of the brain in response to applied focused ultrasound treatment. Additionally or alternatively, the second image can be acquired via diffusion tensor imaging.

At 906, the first image is segmented into a plurality of subregions of the brain to generate a segmented first image, such that each of at least a subset of a plurality of voxels comprising the first image are associated with one of the plurality of subregions. It will be appreciated that not all portions of the first image may be of interest for a given disorder, and thus only those voxels representing the plurality of subregions may be included in the segmentation. At 908, a location within a region of interest of the brain is selected as an initial target for focused ultrasound treatment according to the segmented first image and the second image. It will be appreciated that a given treatment can be performed over a number of individual locations, and that this analysis can be performed for each of those locations.

At 910, focused ultrasound treatment is applied to the selected location and feedback from the patient in response to the applied focused ultrasound treatment is measured at 912. The feedback can include observations of a clinician on the appearance and behavior of the patient, self-reporting from the patient about symptoms of the disorder, measured electrical activity in the brain, and biometric parameters, such as those described in Tables I-VII above. In particular, imaging may be used to determine the effectiveness and potential side effects at a given location. For example, fluid-attenuated inversion recovery (FLAIR) MRI sequences can be indicative of antibody delivery and a degree of blood-brain barrier opening when focused ultrasound treatment includes introduction of antibodies to the brain. If the FLAIR signal at the location is too high, further application of energy the location can avoided for a given treatment. Contrast MRI can be used in a similar fashion. For treatment of neurocognitive disorders, amyloid positron emission tomography (PET) can be used to evaluate reduction of protein at a given location. An additional imaging quantitative feedback is GRE of T2* scans what can show dark spots in a T2* MRI dark spots can indicate damage, and thus a need to avoid further application of energy to the location. Acoustic feedback can also be used in focused ultrasound applications to evaluate both safety and effectiveness of the treatment. These imaging assessments provide quantitative personalized assessments of therapy safety, dose and benefit, and are analogous to digital fingerprinting.

At 914, an effectiveness of the focused ultrasound treatment is determined according to the measured feedback. This can be done, for example, either by a rule-based approach or by providing the measured feedback parameters to a predictive model. If the modulation is determined to be effective (Y), the location of the focused ultrasound treatment is retained for subsequent focused ultrasound treatments at 916 and the method terminates. If not (N), the selected location is determined to be ineffective, and the method returns to 908 to select a new location within the region of interest as a target for focused ultrasound treatment. The location of the focused ultrasound treatment target is personalized and can be modified live during treatment from acute feedback, or for subsequent treatments. The dosing of the focused ultrasound treatment can also be adjusted.

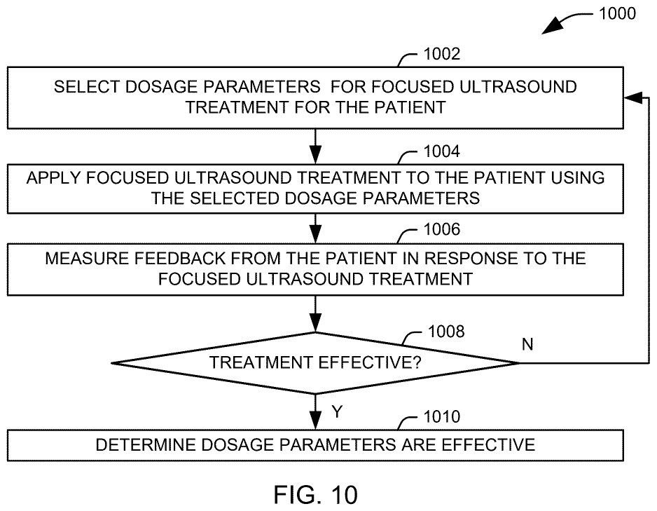

FIG. 10 illustrates a method 1000 for selecting a dose for focused ultrasound treatment. It will be appreciated that the dose for focused ultrasound treatment is defined by parameters such as a frequency of application of the focused ultrasound treatment, an energy profile of the focused ultrasound treatment, a shape of the energy field, a direction of the energy field, a pulse rate of the focused ultrasound, a duration of discrete applications of energy, a number of discrete applications of energy, a total duration of the treatment, a dosage of microbubbles provided to the patient for treatments used to selectively open the blood-brain barrier, a type or dosage of a therapeutic provided in concert with the treatment, and an intensity or power of the treatment. At 1002, an initial dose for the modulation can be selected. It will be appreciated that the initial dose can be standard across patients, but in the illustrated implementation, the initial dose is patient-specific and can be determined according to factors such as a severity of the disorder, a size of the patient's head, the type of tissue targeted, a skull density ratio of the patient, and the location of focused ultrasound treatment relative to the skull. For example, a lower skull density ratio for a patient might require a higher dose (e.g., higher intensity or power), whereas the effects of location can vary with the treatment modality, with locations close to the skull requiring different settings in focused ultrasound, as fewer transducers can effectively target the location.

At 1004, focused ultrasound treatment is applied to the selected location and feedback from the patient in response to the applied focused ultrasound treatment is measured at 1006. The feedback can include observations of a clinician on the appearance and behavior of the patient, self-reporting from the patient about symptoms of the disorder, measured electrical activity in the brain, and biometric parameters, such as those described in Tables I-VII above. In particular, imaging may be used to determine the effectiveness and potential side effects of a given treatment. For example, Fluid-attenuated inversion recovery (FLAIR) MRI sequences can be indicative of antibody delivery and a degree of blood-brain barrier opening when focused ultrasound treatment includes introduction of antibodies to the brain. If the FLAIR signal at the location is too high, the dosage (e.g., power, duration, Microbubble dose, etc) can be reduced for subsequent parts of the treatment or subsequent treatments. Contrast MRI can be used in a similar fashion. For treatment of neurocognitive disorders, amyloid positron emission tomography (PET) can be used to evaluate reduction of protein. Dark spots in a T2* MRI dark spots can indicate damage, and thus a need for a lower dose or different approach. Acoustic feedback can also be used in focused ultrasound applications to evaluate both safety and effectiveness of the treatment. In particular, the size and expansion of microbubbles provided to the patient for allowing the blood-brain barrier to be opened via applied ultrasound can be monitored to ensure the safety and efficacy of treatment.

At 1008, an effectiveness of the focused ultrasound treatment is determined according to the measured feedback. This can be done, for example, either by a rule-based approach or by providing the measured feedback parameters to a predictive model. If the modulation is determined to be effective (Y), the selected dosage parameters for the focused ultrasound treatment are retained for subsequent application of energy at 1010 and the method terminates. If not (N), the selected dosage parameters are determined to be ineffective, and the method returns to 1002 to select new dosage parameters for treatment.

Returning to FIG. 1, once the treatment has been applied, feedback is collected from the patient to determine if the treatment has been effective. It will be appreciated that this can be done during or immediately after treatment (“acute feedback”), a short time (e.g., five hours to five days) after a treatment (“subacute feedback”) or a longer time (e.g., more than five days after a treatment (“chronic feedback”). The feedback can be acquired, for example, as any of the parameters listed in tables I-VII. To collect acute feedback, the tasks can be presented during or immediately after treatment to determine whether the patient's performance increases, decreases or otherwise changes in response to the treatment. The collected feedback can also include self-reporting from the patient, observations by clinicians, measured biometric parameters, such as heart rate variability and blood pressure, and other relevant parameters. In general, the parameters can be adapted based on review by a clinician, a rule-based system that generates suggested changes based on the initial parameters and the measured feedback, or via a predictive model trained on feedback data, treatment parameters, and clinical outcomes for previous patients. One example of the use of feedback in treating disorders, in the context of addiction, can be found in U.S. Published Patent Application No. 2021/0162216, titled Neuromodulatory Methods for Improving Addiction using Multi-dimensional Feedback, the entire contents of which are hereby incorporated by reference.

Acute and subacute feedback from presentation application of a treatment can be obtained, for example, by measuring physiological parameters. In some examples, the physiological parameter can be a response of the patient's autonomic nervous system to focused ultrasound treatment and multiple physiological parameters can be measured during any given assessment session. The physiological parameters can be measured via a wearable device such as a ring, watch, or belt or via a smart phone or tablet, for example, in a naturalistic non-clinical setting such as when the patient is at home, work or other non-clinical setting. Exemplary physiological parameters include heart rate, heart rate variability, perspiration, salivation, blood pressure, pupil size, changes in pupil size, eye movements, brain activity, electrodermal activity, body temperature, and blood oxygen saturation level. Table I, above, provides non-limiting examples of physiological parameters that can be measured and exemplary tests to measure the physiological parameters. Sleep parameters can also be measured using wearable devices.

Particularly in treating neurocognitive disorders, cognitive parameters can be assessed by a battery of cognitive tests that measure, for example, executive function, decision making, working memory, attention, and fatigue. Table II, above, provides non-limiting examples of cognitive parameters that are gamified and that can be measured and exemplary methods and tests/tasks to measure such cognitive parameters. These cognitive tests can be administered in a clinical/laboratory setting or in a naturalistic, non-clinical setting such as when the user is at home, work, or other non-clinical setting. A smart device, such as a smartphone, tablet, or smart watch, can facilitate measuring these cognitive parameters in a naturalistic, non-clinical setting. For example, the Erikson Flanker, N-Back and Psychomotor Vigilance Tasks can be taken via an application on a smart phone, tablet, or smart watch.

Behavioral and psychosocial parameters, such as those described in Table III above, can measure the user's functionality, such as the user's movement via wearable devices as well as subjective/self-reporting questionnaires. The subjective/self-reporting questionnaires can be collected in a clinical/laboratory setting or in a naturalistic, in the wild, non-clinical setting such as when the user is at home, work, or other non-clinical setting. A smart device, such as a smartphone, tablet, or personal computer can be used to administer the subjective/self-reporting questionnaires. Using embedded accelerometers and cameras, these smart devices can also be used to capture the user's movements as well as facial expression analysis to analyze the user's facial expressions that could indicate mood, anxiety, depression, agitation, and fatigue. A wearable or portable device can also be used to measure parameters representing sleep length, sleep depth, a length of a sleep stage, and heart rate variability.

In the illustrated implementation, chronic feedback can be obtained via imaging of the brain and eye, specifically the retina, optic nerve, and associated vasculature, to observe changes relative to the presence or progression of a cognitive disorder. To this end, one or more of a PET scan (including, for example, PET scan using fluorodeoxyglucose (FDG) PET scan, or a PET scan with a radioactive fluorine labeled ligand-linked marker), a gradient recalled echo T2*-weighted imaging (GRE T2*) scan, a, magnetization T1 preparation sequence scan, a T2 weighted fluid attenuation inversion recovery (FLAIR) scan, a susceptibility weighted imaging (SWI) scan, a T1 contrast scan (including, for example, a T1 gadolinium contrast scan), an arterial spin labeling (ASL) scan, and/or a dynamic contrast-enhanced (DCE) MR perfusion scan can be evaluated, either by a clinician or via a predictive model, to determine the effects of the treatment on the patient.

In some examples, where the image is a PET scan, the scan can be analyzed to determine changes in the concentration of beta-amyloid proteins, tau proteins, and/or other biomarkers of a neurodegenerative disorder based on, for example, previous PET scan(s) or predetermined threshold/baseline values for these biomarkers. The therapy can be delivered or adjusted based on the changes in the concentration. For example, the therapy can be adjusted or delivered upon a detection of a reduction of a concentration or density of a beta-amyloid protein, a tau protein, and/or another biomarker of the neurodegenerative disorder. Alternatively, the therapy can be adjusted or delivered upon a detection of no reduction in the concentration/density of the beta-amyloid protein, the tau protein, and/or another biomarker of the neurodegenerative disorder; or upon a detection of a reduction in the concentration/density of the beta-amyloid protein, the tau protein and/or the another biomarker of the neurodegenerative disorder that is insufficient to improve the human patient's neurodegenerative disorder. This determination can be performed acutely, sub-acutely or chronically after providing the focused ultrasound treatment. As such, these signals can provide immediate/acute feedback during treatment, sub-acute feedback after minutes/hours/days of providing therapy and chronically after weeks/month of providing therapy.

At 110, a focused ultrasound treatment is provided to the patient. It will be appreciated that a focused ultrasound treatment can include neuromodulation via focused ultrasound, disruption of the blood-brain barrier via microbubbles provided to the bloodstream of the patient in combination with administration of therapeutics to the bloodstream, or a combination of both neuromodulation and administration of therapeutics with targeted disruption of the blood-brain barrier. In one implementation, administration of neuromodulation can be preceded or accompanied by priming, in which that patient is presented with a stimulus associated with their disorder to increase neural activity in regions of the brain associated with the disorder. For example, a patient with a neurodegenerative order may be asked to perform a cognitive task immediately before or during neuromodulation. A patient suffering from addiction might be presented with a cue associated with their addiction likely to induce or intensify cravings and/or anxiety, such as a smell, taste, image, video, sound, or tactile stimulation. A patient with PTSD, obsessive-compulsive disorder, a phobia, or an anxiety disorder might be shown a cue likely to trigger or intensify symptoms of their disorder, for example a cue linked to the trauma. A patient with depression might be shown a cue intended to invoke or intensify negative effect. A patient with chronic pain might be subjected to a painful stimulus. A patient with Huntington's disease, Parkinson's disease, a stroke, or autism might be presented with a stimulus associated with the cognitive or behavioral defects exhibited by the patient. For example, depth perception is often a problem in Parkinson's patients, and asking the patient to perform a task reliant on depth perception can provide a priming effect for treating that defect.

At 112, the treatment provided to the patient is adjusted based upon measured feedback. In general, the treatment can be adapted based on review of the feedback by a clinician, a rule-based system that generates suggested changes based on the initial parameters and the measured feedback, or via a predictive model trained on feedback data, treatment parameters, and clinical outcomes for previous patients. The feedback after focused ultrasound treatment can influence whether further focused ultrasound treatment is provided and how the provided focused ultrasound treatment should be adjusted. In terms of adjusting therapy in the context of focused ultrasound treatment, methods can involve adjusting the parameters or dosing of the focused ultrasound treatment such as, for example, the duration, frequency, or intensity of the focused ultrasound treatment. When the collected data indicates that the patient's condition has not improved, a method can involve adjusting the focused ultrasound treatment so that the focused ultrasound treatment is more effective. For example, if the patient was previously having focused ultrasound (FUS) delivered for five minutes during a therapy session, the patient can have the FUS subsequently delivered for twenty minutes during each session or if the patient was having FUS delivered every thirty days, the patient can have FUS subsequently delivered every two weeks. Conversely, if the parameter measurements indicate improvement, the focused ultrasound treatment parameters may not need adjustment and subsequent focused ultrasound treatment sessions can serve primarily as maintenance sessions or the intensity, frequency or duration of the focused ultrasound treatment can be decreased, for example. The above scenarios are only exemplary and are provided to illustrate that the presence and type of change of the patient's physiological parameter measurement values during and after therapy can influence whether the therapy should be adjusted or terminated.

Further, the degree of the patient's physiological, cognitive, psychosocial, or behavioral parameter measurement value during or after therapy can influence the parameters of subsequent focused ultrasound treatment. For example, if the specific patient seeking therapy has a physiological, cognitive, psychosocial, or behavioral parameter measurement value during or after treatment that is higher than the average parameter measurement value of the same patient population, the therapy can be more aggressive subsequently. Conversely, if the specific patient's parameter measurement value during or after treatment is lower than the average parameter measurement value of the same patient population, the therapy can be less aggressive subsequently. In other words, the severity or degree of the patient's physiological, cognitive, psychosocial, or behavioral parameter measurement value during or after focused ultrasound treatment (as well as baseline values and levels) can correlate to the degree or aggressiveness of future focused ultrasound treatment. The above scenarios are only exemplary and are provided to illustrate that the degree of change of the patient's physiological parameter measurement values during and after focused ultrasound treatment can influence the parameters of subsequent therapy.

In certain aspects, acute, subacute, and chronic feedback each determined from one or more combinations of a physiological, a cognitive, a psychosocial, and a behavioral parameter of the patient after a treatment. For example, obtaining a measurement of baseline values of one or more combinations of a physiological, a cognitive, a psychosocial, and a behavioral parameter of the patient can be obtained. The patient can then be exposed to an initial focused ultrasound signal to a neural target site of the patient. A subsequent measurement can be obtained of resultant values of the one or more combinations of the physiological, the cognitive, the psychosocial, and the behavioral parameter of the patient during or after application of the initial focused ultrasound signal. The resultant values can be compared to the baseline values to determine if the patient's cognitive and/or behavioral functions has improved. The focused ultrasound treatment can be adjusted upon a determination that the patient's cognitive and/or behavioral functions has not improved. For example, if it is determined that the focused ultrasound treatment was not successful, the focused ultrasound treatment can be provided to a different target location. Once the new treatment parameters have been adjusted, the method returns to 106 to continue monitoring the patient.

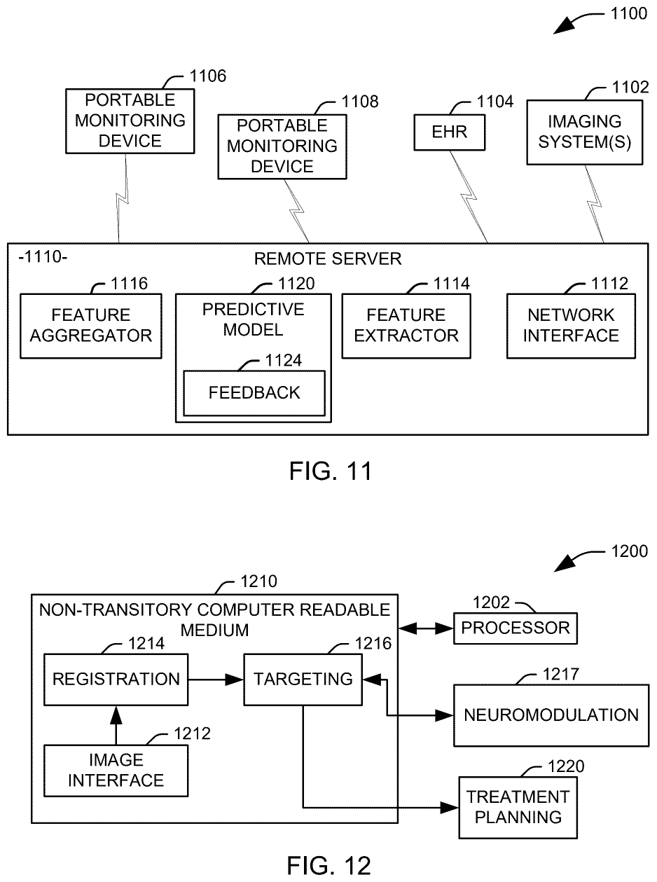

FIG. 11 illustrates a system 1100 for providing treatment to a patient via focused ultrasound treatment. The system 1100 includes a plurality of data sources that provide data to a central server 1110. One or more imaging systems 1102 are configured to provide images of either a brain or an eye of the patient. In practice the imaging systems 1102 can include computed tomography images, magnetic resonance imaging (MRI) systems, positron emission tomography (MRI) systems, optical coherence tomography systems, angiography imagers, and visible and infrared light cameras. The images provided from the one or more imaging systems can be provided directly to the central server 1110 or provided to an electronic health records (EHR) database 1104 that can be accessed by the central server 1110. The portable monitoring devices 1106 and 1108 can include wearable devices that can be used to measure physiological parameters as well as sleep parameters.

The patient can also be provided with portable monitoring devices 1106 and 1108 that include sensors for monitoring systems tracking wellness-relevant parameters for the user. It will be appreciated that a given portable monitoring device (e.g., 1106) can either communicate directly with the central server 1110 to provide the wellness-relevant parameters to the server or with another portable monitoring device (e.g., 1108) that relays the wellness-relevant parameters to the server. In one example, the plurality of portable monitoring devices can include a physiological sensing device and a portable computing device. By using portable monitoring devices 1106 and 1108, measurements can be made continuous from any of a user's home, classroom, job, or sports field-literally anywhere from the battlefield to the board room. Additional parameters can be either retrieved from the EHR database 1104 and/or other available databases via a network interface 1112 associated with the server 1110. These parameters can include, for example, employment information (e.g., title, department, shift), age, sex, home zip code, genomic data, nutritional information, medication intake, household information (e.g., type of home, number and age of residents), social and psychosocial data, consumer spending and profiles, financial data, food safety information, the presence or absence of physical abuse, and relevant medical history.

Between the imaging system or systems 1102, the electronic health database 1104, and the portable monitoring systems 1106 and 1108, each of the parameters listed in Tables I-VII above can be measured or accessed. For example, many behavioral and psychosocial parameters can be determined from the user's functionality and activity as well as subjective/self-reporting questionnaires. The subjective/self-reporting questionnaires can be collected in a clinical/laboratory setting or in a naturalistic, in the wild, non-clinical setting such as when the user is at home, work, or other non-clinical setting. One of the portable monitoring systems 1106 and 1108 can be a smart device, such as a smartphone, tablet, or personal computer that can be used to administer the subjective/self-reporting questionnaires. Using embedded accelerometers and cameras, these smart devices can also be used to capture the facial expression analysis to analyze the user's facial expressions that could indicate mood, anxiety, depression, agitation, and fatigue, as well as measures of the patient's activity. Similarly, cognitive parameters can be determined from tests administered on a smart device. Physiological parameters and sleep parameters can be measured the at portable monitoring systems. Motor and biomarker parameters can be measured in a clinical environment and recorded in the EHR database 1104 for later retrieval, as can any of the physiological, sleep, behavioral, cognitive, and psychosocial parameters that cannot be measured using the portable monitoring device.

The central server 1110 analyzes the data collected by the portable monitoring devices 1106 and 1108 and any clinical data received from the EHR system 1104 and the imaging systems 1102 at the network interface 1112. The central server 1110 can be implemented as a dedicated physical server or as part of a cloud server arrangement. In addition to the remote server, data can be analyzed, in whole or in part, on the local device itself and/or in a federated learning mechanism, in which case, any data from the EHR 1104 can be provided to the local device via an appropriate network interface. Information received from the portable monitoring devices 1106 and 1108 and the network interface 1112, is provided to a feature extractor 1114 that extracts a plurality of features. In one implementation, these features are aggregated at a feature aggregator 1116 to provide a set of aggregate parameters. In this example, the aggregate parameters are then either used directly as features for a predictive model 1120 or used to derive features for the predictive model.

The feature extractor 1114 determines categorical and continuous parameters representing the wellness-relevant parameters. In one example, the parameters can include descriptive statistics, such as measures of central tendency (e.g., median, mode, arithmetic mean, or geometric mean) and measures of deviation (e.g., range, interquartile range, variance, standard deviation, etc.) of time series of the monitored parameters, as well as the time series themselves. Specifically, the feature set as a social provided to the predictive model can include, for at least one parameter, either two values representing the value for the parameter at different times or a single value, such as a measure of central tendency or a measure of deviation which represents values for the parameter across a plurality of times. In addition, the feature set can use features associated with other patients associated with the patient, such as a spouse, child, co-worker, or friend, as additional features.

In other examples, the features can represent departures of the user from an established pattern for the features. For example, values of a given parameter can be tracked overtime, and measures of central tendency can be established, either overall or for particular time periods. The collected features can represent a departure of a given parameter from the measure of central tendency. For example, changes in the activity level of the user, measured by either or both of kinematic sensors and global positioning system (GPS) tracking can be used as a wellness-relevant parameter. Additional elements of monitoring can include the monitoring of the user's compliance with the use of a smart phone, TV, portable device, a portable device. For example, a user may be sent messages by the system inquiring on their wellness level, general mood, or the status of any other wellness-relevant parameter on the portable computing device. A measure of compliance can be determined according to the percentage of these messages to which the user responds via the user interface on the portable computing device, and this measure of compliance can be used as a feature for the predictive model 1120.

In one implementation, the feature extractor 1114 can perform a wavelet transform on a time series of values for one or more parameters to provide a set of wavelet coefficients. It will be appreciated that the wavelet transform used herein is two-dimensional, such that the coefficients can be envisioned as a two-dimensional array across time and either frequency or scale.

The feature extractor 1114 can also include a facial expression classifier (not shown) that evaluates recorded data from a camera and/or recorded images or videos of the user's face from one of the portable monitoring devices 1106 and 1108, such as a smartphone or other mobile device, to assign an emotional state to the user at various times throughout the day. The extracted features can be categorical, representing the most likely emotional state of the user, or continuous, for example, as a time series of probability values for various emotional states (e.g., anxiety, discomfort, anger, etc.) as determined by the facial expression classifier. The feature extractor 1114 can also include one or more image classifiers that reduce provided medical images from the imager systems 1102 to categorical or continuous features for use at the predictive model. It will be appreciated that each of the facial expression classifier and the one or more image classifiers can be implemented using one or more of the models discussed above for use in a predictive model. In one example, the feature extractor 1114 can provide the medical images to a convolutional neural network (CNN) and extract one or more latent values from the CNN as features.

In the example using aggregate features, the feature aggregator 1116 generates a set of aggregate parameters from the set of wellness-relevant parameters collected by the portable monitoring devices 1106 and 1108 and any clinical data received from the EHR system 1104 at the network interface 1112. It will be appreciated that each aggregate parameter can be a weighted combination of the set of wellness-relevant parameters or functions of parameters from the set of wellness-relevant parameters. Accordingly, a given aggregate parameter can represent a plurality of wellness-relevant parameters, and, in general, the plurality of wellness-relevant parameters represented by each aggregate parameter will be related, such that the aggregate parameter represents a specific domain of wellness for the user. In general, each aggregate parameters can use parameters from various sources, such that a given aggregate parameter can be a combination of features from two or more of the portable monitoring devices 1106 and 1108 and the network interface 1112. In some implementations, the system 1100 can include multiple predictive models (not shown) that each receive a unique proper subset of the aggregate parameters. Each predictive model can provide a different clinical parameter representing a different aspect of the user's wellness, such that the aggregate parameters can be utilized for multiple purposes in evaluating the wellness of the user.

The predictive model 1120 can utilize one or more pattern recognition algorithms, each of which analyze the extracted features or a subset of the extracted features to assign a continuous or categorical clinical parameter to the user. It will be appreciated that the predictive model 1120 can use any of the various pattern recognition algorithms described above for use with predictive models. In one example, the predictive model 1120 can assign a continuous parameter that corresponds to a likelihood that that user has or is at risk for a disorder, a likelihood that the user is experiencing the effects of aging, a likelihood that the user is experiencing an onset of dementia, a likelihood that the user has or will develop a neurodegenerative disorder, a likelihood that the user will experience an intensifying of symptoms, or “flare-up,” of a chronic condition, such as chronic pain or anxiety, a likelihood that the user will use an addictive substance during rehabilitation or treatment, a current or predicted level of pain for the user, an expected performance level of the user associated with a current or future time for a particular activity or occupation, a change in symptoms associated with a disease or disorder, a current or predicted response to treatment, a likelihood that the user has experienced an increase in stress, or an overall wellness level of the user. It will be appreciated that the specific nature of the clinical parameter assigned by the predictive model will vary with the problem to which the model is being applied, such as screening, symptom detection or prediction, or determining the effectiveness of applied focused ultrasound treatment.

In another example, the predictive model 1120 can assign a categorical parameter that corresponds to ranges of the likelihoods described above, the presence or risk of a specific disorder, a set of categories representing the users readiness for a particular activity or occupation, categories representing changes in symptoms associated with a disease or disorder (e.g., “improving”, “stable, “worsening”), categories representing a current or predicted response to treatment, categories representing a status of the user (e.g., normal,” “stressed”, “ill”), or categories indicating that a particular action should be suggested to the user. The generated parameter can be stored in a non-transitory computer readable medium, for example, as part of a record in an electronic health records database, or used to suggest a course of action to the user.

In one implementation, the predictive model 1120 can include a constituent model that predicts future values for the wellness-related parameters or aggregate parameters, such as a convolutional neural network that is provided with one or more two-dimensional arrays of wavelet transform coefficients as an input. The wavelet coefficients detect changes not only in time, but also in temporal patterns, and can thus reflect changes in the ordinary biological rhythms of the user. Additionally, or alternatively, the predictive model can use constituent models that predict current or future values for the wellness-related or aggregate parameters, with these measures then used as features for generating the output of the predictive model. This data can also be used to group the user with users who respond similarly to these parameters, with data fed back from users within a given group used to better tailor the model to the user.

The predictive model 1120 can also be used to facilitate a feedback strategy to one or more designated recipients, which can include the user, a health care provider, family, friends, social system, a care team, a supervisor, a coach, a caretaker, and other entities, in which a course of action is suggested, for example, in response to a change in the wellness of the individual. Examples of disorders for which detection or the disorder or a heightened risk of the disorder can trigger intervention include, but are not limited to, anxiety, depression, suicidal thoughts, stress, mood, agitation, obsessions, compulsion, OCD, Parkinson's, tremor, and chronic pain. The feedback or intervention can be include feedback from the system for raising awareness of a detected issue or education on that issue, initiation or modification of a current treatment including but not limited to focused ultrasound treatment, medications, biologicals, surgical intervention, behavioral and social intervention, digital intervention via portable device, a care provider coming to the individual, directing an individual to go to a clinic, support group, emergency room, or hospital with provided directions or a location for the clinic, support group, emergency room, or hospital, or directing the user to obtain additional testing.

It will be appreciated that a suggested course of action can be any course of action intended to enhance the wellness of the user or others and can include, for example, taking a prescribed meditation, performance of prescribed exercises, cessation of a current activity, and contacting a medical professional. In one example, when the user is determined to be experiencing significant stress, the feedback can include a “digital reset” in which the user is instructed to engage in deep breathing or other stress reduction techniques. If this is ineffective or if the problem reoccurs, the feedback can be elevated to a digital intervention prescribed by a medical expert, in which the user engages in guided stress reduction techniques as periodic intervals. If this is also ineffective, the user can be instructed to seek the assistance of a counselor, coach, or therapist. It will be appreciated that the model is predictive, and thus interventions for stress, pain, and similar issues can be suggested before the user is even aware of the issue. In another example, a user can be instructed in sleep hygiene in response to indications of inadequate or restless sleep, directed to engage in a sleep study, or referred to a physician for analysis. It will be appreciated that interventions associated with sleep can also be assigned for other detected issues, such as decreased cognitive function, decreased athletic, job, or other performance, or heightened risks of stroke and heart disease.

In some implementations, the predictive model 1120 can include a feedback component 1124 can tune various parameters of the predictive model 1120 based upon the accuracy of predictions made by the model. In one example, the feedback component 1124 can be shared by a plurality of predictive models 1120, with the outcomes for users associated with each predictive model compared to the outcomes predicted by the output of the model. Parameters associated with the model, such as thresholds for producing categorical inputs or outputs from continuous values or parameters associated with the pattern recognition algorithms comprising the predictive model, can be adjusted according to the differences in the actual and predicted outcomes.

Alternatively, the predictive model 1120 can obtain feedback at the level of the individual model. For example, in a predictive model 1120 using constituent models to predict future values of wellness-relevant parameters, the model receives consistent feedback as to the accuracy of these predictions once the wellness-relevant parameter is measured. This feedback can be used to adjust parameters of the model, including individualized thresholds for that user to produce categorical inputs or outputs from continuous values, or baseline values for biological rhythms associated with the user. Alternatively, feedback can be provided from a final output of the model and compared to other data, such as a user-reported status, to provide feedback to the model. In one implementation, a reinforcement learning approach can be used to adjust the model parameters based on the accuracy of either predicted future values of wellness-relevant parameters at intermediate stages of the predictive model 1120 or the output of the predictive model. For example, a decision threshold used to generate a categorical output from a continuous index produced by the predictive model 1120 can be set at an initial value based on feedback from a plurality of models from previous users and adjusted via the reinforcement model to generate a decision threshold specific to the user.

FIG. 12 illustrates a system 1200 for targeting focused ultrasound treatment for treatment or diagnosis of a disorder to a specific region of the brain of a patient. The system 1200 includes a processor 1202 and a non-transitory computer readable medium 1210 that stores executable instructions for targeting focused ultrasound treatment for treatment or diagnosis of disorders. The executable instructions include an imaging interface 1212 that receives a first image, representing a structure of the brain, and a second image, representing a connectivity of the brain, for a patient from one or more associated imaging systems (not shown). In one implementation, the first image is a T1 magnetic resonance imaging (MRI) image, and the second image is a diffusion tensor imaging (DTI) image generated using an MRI imager. The imaging interface 1212 can include appropriate software components for communicating with an imaging system (not shown) or repository of stored images (not shown) over a network via a network interface (not shown) or via a bus connection.

The first image is provided to a registration component 1214 that segments the first image into a plurality of subregions of the brain. The identified subregions can include, for example, a frontal pole, a temporal pole, a superior frontal region, a medial orbito-frontal region, a caudal anterior cingulate, a rostral anterior cingulate, an entorhinal region, a parahippocampal region, a peri-calcarine region, a lingual region, a cuneus region, an isthmus region, a pre-cuneus region, a paracentral lobule, and a fusiform region. In one example, the registration component 1214 registers the first image to a standard atlas to provide the segmentation. In another implementation, a convolutional neural network, trained on a plurality of annotated image samples, can be used to provide the segmented image. One example of such a system can be found in 3D Whole Brain Segmentation using Spatially Localized Atlas Network Tiles, by Huo et al. (available at https://doi.org/10.48550/arxiv. 1903.12152), which is hereby incorporated by reference in its entirety. The registration component 1214 can also register the second image with the first image, such that the location of nodes within the connectome within the brain is known.

Each of the segmented first image and the second image can be provided to a targeting component 1216 that selects a location and intensity profile for the focused ultrasound treatment. It will be appreciated that the segmented first image can be registered with the second image before it is provided to the targeting component. The targeting component 1216 generates a connectome of the brain, representing neural connections within the brain, from the second image. A region of interest can be defined within the first image based upon the known subregions, and the location and intensity profile of the focused ultrasound treatment within the region of interest can be selected according to the generated connectome. It will be appreciated that the connectome can be determined as a passive connectome, representing the physical connectivity among portions of the brain, or an active connectome, representing the activity induced in portions of the brain in response to energy provided in a specific location.

In one example, the targeting component 1216 can operate in conjunction with a focused ultrasound treatment system 1217 to generate a map of an active connectome of the brain. Specifically, energy can be applied at various locations within the region of interest, and activity within the brain can be determined via an appropriate functional imaging modality. In one implementation, the activity measured within the brain in response to each location is recorded as a result of focused ultrasound treatment for that location. It will be appreciated that multiple locations can be impacted when energy is provided, and the detected activity can be attributed to each location, for example, represented as voxels, according to the percentage of energy received at each voxel. Alternatively, the results of multiple measurements can be compared, for example, via solving for one or more n-dimensional linear systems, where n is the number of voxels within the region of interest. Accordingly, the active connectivity associated with each location within the region of interest can be determined.

In one implementation, the region of interest can be divided into a set of voxels, and each voxel can be assigned a cost based upon its connection to other regions of the brain in the connectome to form a cost map. For example, a positive cost can represent representing a location within the region of interest that is connected to portions of the brain for which, taken in aggregate, stimulation is not desirable, and a negative cost can represent a location within the region of interest that is connected to portions of the brain for which, taken in aggregate, stimulation is desirable. It will be appreciated that this can be reversed to instead create a “utility map,” with positive values representing locations for which stimulation is desirable.

Each location has an associated intensity profile around a reference point, such as a center point, representing an amount of energy provided to the region for a given location of the reference point. In one example, each voxel can be assigned a value normalized by a maximum intensity, and this value can be used to weight the contribution of the voxel to the overall cost associated with the location and intensity profile. An optimization process, such as gradient decent, can be used to search the region of interest for an optimal or near-optimal location and intensity profile, and the resulting location and intensity profile can be provided to a treatment planning system 1220 for use in generating a treatment plan for the patient.

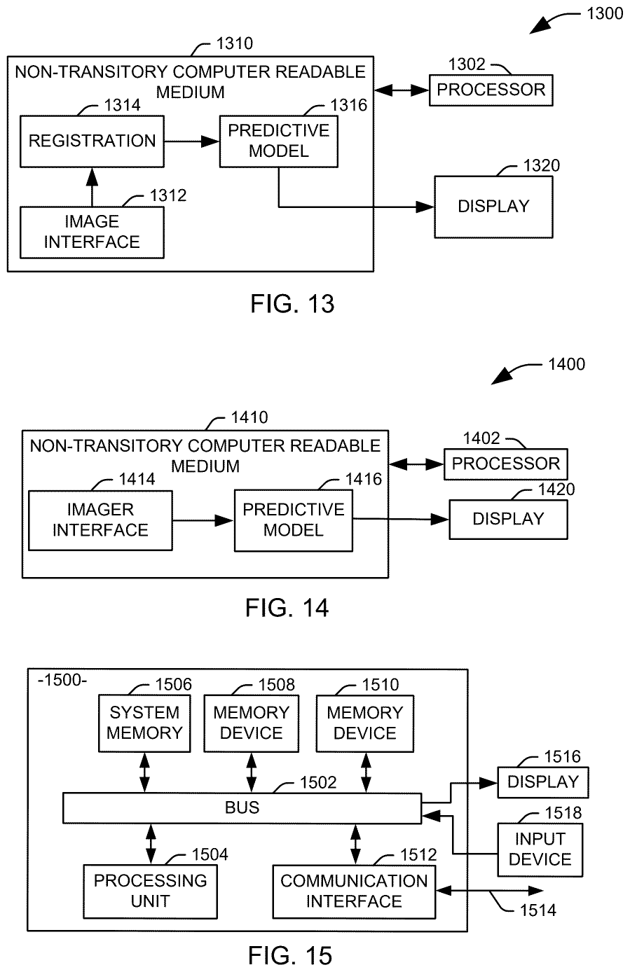

FIG. 13 illustrates a system 1300 for determining a presence or risk of a disorder from imaging of a brain of a patient. The system 1300 includes a processor 1302 and a non-transitory computer readable medium 1310 that stores executable instructions for determining a risk or presence of a disorder from imaging of a brain of a patient. The executable instructions include an imaging interface 1312 that receives a first image, representing a structure of the brain, and a second image, representing a connectivity of the brain, for a patient. In one implementation, the first image is a T1 magnetic resonance imaging (MRI) image, and the second image is a diffusion tensor imaging (DTI) image generated using an MRI imager. The imaging interface 1312 can also receive functional images, for example, from a same or a different MRI imager, representing activity within the brain. The imaging interface 1312 can include appropriate software components for communicating with an imaging system (not shown) or repository of stored images (not shown) over a network via a network interface (not shown) or via a bus connection.

The first image is provided to a registration component 1314 that segments the first image into a plurality of subregions of the brain. The identified subregions can include, for example, a frontal pole, a temporal pole, a superior frontal region, a medial orbito-frontal region, a caudal anterior cingulate, a rostral anterior cingulate, an entorhinal region, a parahippocampal region, a peri-calcarine region, a lingual region, a cuneus region, an isthmus region, a pre-cuneus region, a paracentral lobule, and a fusiform region. In one example, the registration component 1314 registers the first image to a standard atlas to provide the segmentation. In another implementation, a convolutional neural network, trained on a plurality of annotated image samples, can be used to provide the segmented image. The registration component 1314 registers the second image with the first image, such that the location of nodes within the connectome within the brain is known.

The registered second image is provided to a predictive model 1316. The predictive model 1316 analyzes a provided connectome image to assign a clinical parameter to the user representing one of a likelihood that a patient has or will have issues within a specified time period with a general class of disorders, a likelihood that a patient has or will have issues within a specified time period with a disorder, a likelihood that a patient will respond to treatment for a disorder generally, or a likelihood that the patient will respond to a specific treatment, such as focused ultrasound treatment, for a disorder. It will be appreciated that the clinical parameter can be categorical or continuous. The predictive model 1316 can utilize one or more pattern recognition algorithms, implemented, for example, as classification and regression models as described above. Regardless of the specific model employed, the clinical parameter generated at the predictive model 1316 can be provided to a user at the display 1320 via a user interface or stored on the non-transitory computer readable medium 1310, for example, in an electronic medical record associated with the patient.

FIG. 14 illustrates one example of a system 1400 for diagnosis and monitoring of disorders. The system 1400 includes a processor 1402 and a non-transitory computer readable medium 1410 that stores executable instructions for receiving data representing a patient from a plurality of sources and determining a clinical parameter representing a likelihood that the patient has or will develop a cognitive disorder. The executable instructions include an imaging interface 1412 that receives a first image, representing a structure or connectivity of a brain of the patient, and a second image, representing a one of the retina, the optic nerve, and the associated vasculature of the patient, from respective imaging systems (not shown). In one implementation, the first image is a T1 magnetic resonance imaging (MRI) image representing a structure of the brain. In other implementations, the first image can be a diffusion tensor imaging (DTI) image generated using an MRI imager, a position emission tomography (PET) image acquired using glucose tagged with radioactive fluorine or a tracer for beta-amyloid or the tau protein. The second image can be any image from which the retina, optical nerve, or associated vasculature can be extracted, for example, an optical coherence tomography (OCT) image, an OCT angiography image, and an image acquired via fundus photography. In some examples, the imager interface 1412 can also receive an image or video of a pupil of the patient to provide a parameter representing at least one of eye tracking data, eye movement, pupil size, and a change in pupil size.

The imaging interface 1412 can include appropriate software components for communicating with an imaging system (not shown) or repository of stored images (not shown) over a network via a network interface (not shown) or via a bus connection. In some implementations, the imaging interface 1412 can segments the first image into a plurality of subregions of the brain, such that each of at least a portion of the pixels or voxels comprising the image are associated with one of the plurality of subregions. The identified subregions can include, for example, a frontal pole, a temporal pole, a superior frontal region, a medial orbito-frontal region, a caudal anterior cingulate, a rostral anterior cingulate, an entorhinal region, a parahippocampal region, a peri-calcarine region, a lingual region, a cuneus region, an isthmus region, a pre-cuneus region, a paracentral lobule, and a fusiform region. In one example, the imaging interface 1412 registers the first image to a standard atlas to provide the segmentation. In another implementation, a convolutional neural network, trained on a plurality of annotated image samples, can be used to provide the segmented image. Where multiple images representing the brain taken in different imaging modalities are provided, the imager interface 1412 can register the images with one another. For example, an image representing the structure can be registered with an image representing brain connectivity such that the location of nodes within the connectome within the brain is known.

A predictive model 1416 receives a representation of the first image and a representation of the second image and generates a value representing a clinical property associated with one or more disorders for the patient. For example, the generated value is a categorical or continuous value that represents a likelihood that the patient currently has a disorder. In another example, the generated value is a categorical or continuous value that represents a likelihood that the patient will develop a disorder. In a yet another, the generated value is a categorical or continuous value that represents a likelihood that the patient will respond to a specific treatment for a disorder. In a further example, the generated value is a categorical value that represents an expected best treatment, a best target location, or best set of parameters for a treatment for a disorder for the patient. In a still further example, the generated value is a continuous or categorical value that represents a progression of a disorder for the patient, either in general, or specifically in response to an applied treatment, such as focused ultrasound treatment.

The representation of each of the first image and the second image provided to the predictive model 1416 can include any of the images themselves, represented as chromaticity values from the pixels or voxels comprising the image, images or masks derived from the images, or sets of numerical features extracted from the image. For example, the representation of the first image can be any of a representation of a cortical profile of the brain, a representation of a vasculature of the brain, a representation of a B-amyloid profile of the brain, and a representation of a connectivity of the brain from the first image. It will be appreciated that the predictive model 1416 can also receive clinical parameters representing the patient, for example, measured via one or more sensors and/or retrieved from a medical health records database, such that the value representing a clinical property associated with one or more cognitive disorders for the patient is calculated from the representation of the first image, the representation of the second image, and the clinical parameters.

The predictive model 1416 can utilize one or more pattern recognition algorithms, implemented, for example, as classification and regression models, each of which analyze the provided data to assign the value to the user. It will be appreciated that the predictive model 1416 can use any of the pattern recognition algorithms described above for use in predictive models. Regardless of the specific model employed, the categorical or continuous value generated at the predictive model 1416 can be provided to a user at the display 1420 via a user interface or stored on the non-transitory computer readable medium 1410, for example, in an electronic medical record associated with the patient.

FIG. 15 is a schematic block diagram illustrating an exemplary system 1500 of hardware components capable of implementing examples of the systems and methods disclosed herein. The system 1500 can include various systems and subsystems. The system 1500 can be a personal computer, a laptop computer, a workstation, a computer system, an appliance, an application-specific integrated circuit (ASIC), a server, a server BladeCenter, a server farm, etc.

The system 1500 can include a system bus 1502, a processing unit 1504, a system memory 1506, memory devices 1508 and 1510, a communication interface 1512 (e.g., a network interface), a communication link 1514, a display 1516 (e.g., a video screen), and an input device 1518 (e.g., a keyboard, touch screen, and/or a mouse). The system bus 1502 can be in communication with the processing unit 1504 and the system memory 1506. The additional memory devices 1508 and 1510, such as a hard disk drive, server, standalone database, or other non-volatile memory, can also be in communication with the system bus 1502. The system bus 1502 interconnects the processing unit 1504, the memory devices 1506-1510, the communication interface 1512, the display 1516, and the input device 1518. In some examples, the system bus 1502 also interconnects an additional port (not shown), such as a universal serial bus (USB) port.

The processing unit 1504 can be a computing device and can include an application-specific integrated circuit (ASIC). The processing unit 1504 executes a set of instructions to implement the operations of examples disclosed herein. The processing unit can include a processing core.

The additional memory devices 1506, 1508, and 1510 can store data, programs, instructions, database queries in text or compiled form, and any other information that may be needed to operate a computer. The memories 1506, 1508 and 1510 can be implemented as computer-readable media (integrated or removable), such as a memory card, disk drive, compact disk (CD), or server accessible over a network. In certain examples, the memories 1506, 1508 and 1510 can comprise text, images, video, and/or audio, portions of which can be available in formats comprehensible to human beings.

Additionally or alternatively, the system 1500 can access an external data source or query source through the communication interface 1512, which can communicate with the system bus 1502 and the communication link 1514.

In operation, the system 1500 can be used to implement one or more parts of a system for providing focused ultrasound treatment to a patient in accordance with the present invention. Computer executable logic for implementing the system resides on one or more of the system memory 1506, and the memory devices 1508 and 1510 in accordance with certain examples. The processing unit 1504 executes one or more computer executable instructions originating from the system memory 1506 and the memory devices 1508 and 1510. The term “computer readable medium” as used herein refers to a medium that participates in providing instructions to the processing unit 1504 for execution. This medium may be distributed across multiple discrete assemblies all operatively connected to a common processor or set of related processors. Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to, portable or fixed storage devices, optical storage devices, wireless channels, and/or various other storage mediums capable of storing that contain or carry instruction(s) and/or data.

### Example I

The following procedure is implemented to demonstrate the efficacy of FUS neuromodulation of the subthalamic nucleus to improve visual-spatial perception, depth perception, visual discrimination, working memory, and spatial memory of complex visual stimuli in a human subject with Parkinson's disease.

### I. Patient Preparation and Targeting

Pre-operative MRI imaging is used to identify the stereotactic coordinates of the subthalamic nucleus.

### II. Methodology and Stimuli

The patient is positioned before a display screen. A baseline assessment is conducted where the patient is presented with a battery of visual stimuli designed to test:
Visual working memory, attentional filtering, and visual transition: Visual change detection test. A visual change detection test is a cognitive task where the patient identifies differences between two similar visual scenes or stimuli to measure visual working memory capacity. A specific method includes a “flicker paradigm,” which uses brief disruptions between identical images to reveal missed changes, and “one-shot” tasks where a single probe is compared to an initial display, revealing how many items can be held in short-term memory.
Numerosity and Visual salience: Human figure vs non-human figure numerosity task. A human figure vs. non-human figure numerosity task compares how easily people estimate the number of people (humans) versus objects.
Visuoperceptual interference: Pareidolia test. A pareidolia test is a neuropsychological tool using ambiguous visual noise designed to measure how often the patient perceives meaningful patterns (like faces) in randomness by detecting heightened illusory responses that signal visual hallucinations. While normal pareidolia is common (seeing faces in clouds), the test quantifies abnormal levels, distinguishing between healthy individuals and a patient with Parkinson where such illusions ca be more frequent and intense.
Visual Working memory: Visual object N-Back. A visual object N-back is a cognitive test measuring working memory by showing a sequence of different objects (shapes, pictures) and asking the patient to identify if the current object matches the one shown ‘n’ trials (e.g., 1-back, 2-back) earlier in the sequence, training the ability to hold and update visual information, crucial for attention, learning, and cognitive flexibility.

| Cognitive Construct | | |
|---|---|---|
| Being Assessed | Task | Metric Detected |
| I. Capacity | Change | Visual Working Memory |
| | Detection | (VWM) |
| II. Precision | Numerosity | Approximate Number |
| | | System (ANS) |
| III. Accuracy | Pareidolia | Noise-Signal Discrimination |
| IV. Fluidity | Visual n-Back | Working Memory Updating |

After the baseline assessments, initial FUS neuromodulation is delivered while the patient carries out one or more of the tasks listed above. Parameters for initial FUS neuromodulation are: for a frequency of 220 kilohertz (kHz); a power of 50-100 watts (W); a duty cycle of 3.3% per subspot; 100 repetitions; a repetition time (TR)=3 seconds; a pulse duration of 100 milliseconds (ms); for up to 30 minutes (mins) in six 5 min sonication blocks for one treatment (this equates to estimated root mean square pressure of in situ peak rarefactional pressure of 1.3-2.4 megapascal (MPa)). For a system with a frequency of 500 kHz; a pulse rate of 0.33; a duty cycle of 1.67%; pulse duration=50 ms; for up to 30 mins in six 5 min sonication blocks for one treatment (this equates to in situ peak rarefactional pressure of 1.27-2.2 MPa). Once the initial FUS neuromodulation sonication block is over, the patient carries out identical task assessment as the baseline task assessment. FUS neuromodulation parameters are adjusted based on the comparison of post-sonication block task assessment and the baseline task assessment. If the patient shows improvements in detection of changes during the change detection task, closer estimates of human figure presentations in the numerosity task, reduction in perception of faces and objects in the pareidolia task, and/or improvements in working memory capacity in the visual n-back test, the patient receives additional FUS neuromodulation sonication sessions up to 30 min of total sonication (six 5 min blocks) with the same neuromodulation parameters. If the patient does not show improvements in detection of changes during the change detection task, closer estimates of human figure presentations in the numerosity task, reduction in perception of faces and objects in the pareidolia task, and/or improvements in working memory capacity in the visual n-back test, subsequent FUS neuromodulation parameters are adjusted by increasing the sonication energy until reaching the maximum level for the specific device for the total 30 duration. For the 220 kHz system, the initial sonication block starts with a FUS neuromodulation dose of 50 W for 5 min and if there are no clinically relevant changes in visual spatial assessments then 60 W is used for the second 5 min block. If there are still no clinically relevant improvements, then 70 W is used for the third 5 min block. If there are still no clinically relevant improvements, then 80 W is used for the fourth block. If still yet no clinically relevant improvements occur, then 90 W is used for the fifth block. If there are still no clinically relevant improvements, then 100 W is used.

### III. Results

The application of FUS to the subthalamic nucleus results in an immediate and measurable improvement in task performance compared to baseline and sham conditions.

Specifically, the patient shows:
1. Improvements in detection of changes during the change detection task
2. Closer estimates of human figure presentations in the numerosity task
3. Reduction in perception of faces and objects in the pareidolia task
4. Improvements in working memory capacity in the visual n-back task

These results demonstrate that FUS neuromodulation of the subthalamic nucleus can enhance the processing of visuospatial information, thereby bypassing or compensating for visuospatial deficits from Parkinson's disease.

### Example II

### I. Patient Preparation and Targeting

Pre-operative MRI imaging is used to identify the stereotactic coordinates of the globus pallidus.

### II. Methodology and Stimuli

The patient is positioned before a display screen. A baseline assessment is conducted where the patient is presented with a battery of visual stimuli designed to test:
Visual working memory, attentional filtering, and visual transition: Visual change detection test. A visual change detection test is a cognitive task where the patient identifies differences between two similar visual scenes or stimuli to measure visual working memory capacity. A specific method includes a “flicker paradigm,” which uses brief disruptions between identical images to reveal missed changes, and “one-shot” tasks where a single probe is compared to an initial display, revealing how many items can be held in short-term memory.
Numerosity and Visual salience: Human figure vs non-human figure numerosity task. A human figure vs. non-human figure numerosity task compares how easily people estimate the number of people (humans) versus objects.
Visuoperceptual interference: Pareidolia test. A pareidolia test is a neuropsychological tool using ambiguous visual noise designed to measure how often the patient perceives meaningful patterns (like faces) in randomness by detecting heightened illusory responses that signal visual hallucinations. While normal pareidolia is common (seeing faces in clouds), the test quantifies abnormal levels, distinguishing between healthy individuals and a patient with Parkinson where such illusions ca be more frequent and intense.
Visual Working memory: Visual object N-Back. A visual object N-back is a cognitive test measuring working memory by showing a sequence of different objects (shapes, pictures) and asking the patient to identify if the current object matches the one shown ‘n’ trials (e.g., 1-back, 2-back) earlier in the sequence, training the ability to hold and update visual information, crucial for attention, learning, and cognitive flexibility.

| Cognitive Construct | | |
|---|---|---|
| Being Assessed | Task | Metric Detected |
| I. Capacity | Change | Visual Working Memory |
| | Detection | (VWM) |
| II. Precision | Numerosity | Approximate Number |
| | | System (ANS) |
| III. Accuracy | Pareidolia | Noise-Signal Discrimination |
| IV. Fluidity | Visual n-Back | Working Memory Updating |

After the baseline assessments, initial FUS neuromodulation is delivered while the patient carries out one or more of the tasks listed above. Parameters for initial FUS neuromodulation are: for a frequency of 220 kilohertz (kHz); a power of 50-100 watts (W); a duty cycle of 3.3% per subspot; 100 repetitions; a repetition time (TR)=3 seconds; a pulse duration of 100 milliseconds (ms); for up to 30 minutes (mins) in six 5 min sonication blocks for one treatment (this equates to estimated root mean square pressure of in situ peak rarefactional pressure of 1.3-2.4 megapascal (MPa)). For a system with a frequency of 500 kHz; a pulse rate of 0.33; a duty cycle of 1.67%; pulse duration=50 ms; for up to 30 mins in six 5 min sonication blocks for one treatment (this equates to in situ peak rarefactional pressure of 1.27-2.2 MPa). Once the initial FUS neuromodulation sonication block is over, the patient carries out identical task assessment as the baseline task assessment. FUS neuromodulation parameters are adjusted based on the comparison of post-sonication block task assessment and the baseline task assessment. If the patient shows improvements in detection of changes during the change detection task, closer estimates of human figure presentations in the numerosity task, reduction in perception of faces and objects in the pareidolia task, and/or improvements in working memory capacity in the visual n-back test, the patient receives additional FUS neuromodulation sonication sessions up to 30 min of total sonication (six 5 min blocks) with the same neuromodulation parameters. If the patient does not show improvements in detection of changes during the change detection task, closer estimates of human figure presentations in the numerosity task, reduction in perception of faces and objects in the pareidolia task, and/or improvements in working memory capacity in the visual n-back test, subsequent FUS neuromodulation parameters are adjusted by increasing the sonication energy until reaching the maximum level for the specific device for the total 30 duration. For the 220 kHz system, the initial sonication block starts with a FUS neuromodulation dose of 50 W for 5 min and if there are no clinically relevant changes in visual spatial assessments then 60 W is used for the second 5 min block. If there are still no clinically relevant improvements, then 70 W is used for the third 5 min block. If there are still no clinically relevant improvements, then 80 W is used for the fourth block. If still yet no clinically relevant improvements occur, then 90 W is used for the fifth block. If there are still no clinically relevant improvements, then 100 W is used.

### III. Results

The application of FUS to the globus pallidus results in an immediate and measurable improvement in task performance compared to baseline and sham conditions.

Specifically, the patient shows:
1. Improvements in detection of changes during the change detection task
2. Closer estimates of human figure presentations in the numerosity task
3. Reduction in perception of faces and objects in the pareidolia task
4. Improvements in working memory capacity in the visual n-back task

These results demonstrate that FUS neuromodulation of the globus pallidus can enhance the processing of visuospatial information, thereby bypassing or compensating for visuospatial deficits from Parkinson's disease.

### Example III

### I. Patient Preparation and Targeting

Pre-operative MRI imaging is used to identify the stereotactic coordinates of the putamen.

### II. Methodology and Stimuli

The patient is positioned before a display screen. A baseline assessment is conducted where the patient is presented with a battery of visual stimuli designed to test:
Visual working memory, attentional filtering, and visual transition: Visual change detection test. A visual change detection test is a cognitive task where the patient identifies differences between two similar visual scenes or stimuli to measure visual working memory capacity. A specific method includes a “flicker paradigm,” which uses brief disruptions between identical images to reveal missed changes, and “one-shot” tasks where a single probe is compared to an initial display, revealing how many items can be held in short-term memory.
Numerosity and Visual salience: Human figure vs non-human figure numerosity task. A human figure vs. non-human figure numerosity task compares how easily people estimate the number of people (humans) versus objects.
Visuoperceptual interference: Pareidolia test. A pareidolia test is a neuropsychological tool using ambiguous visual noise designed to measure how often the patient perceives meaningful patterns (like faces) in randomness by detecting heightened illusory responses that signal visual hallucinations. While normal pareidolia is common (seeing faces in clouds), the test quantifies abnormal levels, distinguishing between healthy individuals and a patient with Parkinson where such illusions ca be more frequent and intense.
Visual Working memory: Visual object N-Back. A visual object N-back is a cognitive test measuring working memory by showing a sequence of different objects (shapes, pictures) and asking the patient to identify if the current object matches the one shown ‘n’ trials (e.g., 1-back, 2-back) earlier in the sequence, training the ability to hold and update visual information, crucial for attention, learning, and cognitive flexibility.

| Cognitive Construct | | |
|---|---|---|
| Being Assessed | Task | Metric Detected |
| I. Capacity | Change | Visual Working Memory |
| | Detection | (VWM) |
| II. Precision | Numerosity | Approximate Number |
| | | System (ANS) |
| III. Accuracy | Pareidolia | Noise-Signal Discrimination |
| IV. Fluidity | Visual n-Back | Working Memory Updating |

After the baseline assessments, initial FUS neuromodulation is delivered while the patient carries out one or more of the tasks listed above. Parameters for initial FUS neuromodulation are: for a frequency of 220 kilohertz (kHz); a power of 50-100 watts (W); a duty cycle of 3.3% per subspot; 100 repetitions; a repetition time (TR)=3 seconds; a pulse duration of 100 milliseconds (ms); for up to 30 minutes (mins) in six 5 min sonication blocks for one treatment (this equates to estimated root mean square pressure of in situ peak rarefactional pressure of 1.3-2.4 megapascal (MPa)). For a system with a frequency of 500 kHz; a pulse rate of 0.33; a duty cycle of 1.67%; pulse duration=50 ms; for up to 30 mins in six 5 min sonication blocks for one treatment (this equates to in situ peak rarefactional pressure of 1.27-2.2 MPa). Once the initial FUS neuromodulation sonication block is over, the patient carries out identical task assessment as the baseline task assessment. FUS neuromodulation parameters are adjusted based on the comparison of post-sonication block task assessment and the baseline task assessment. If the patient shows improvements in detection of changes during the change detection task, closer estimates of human figure presentations in the numerosity task, reduction in perception of faces and objects in the pareidolia task, and/or improvements in working memory capacity in the visual n-back test, the patient receives additional FUS neuromodulation sonication sessions up to 30 min of total sonication (six 5 min blocks) with the same neuromodulation parameters. If the patient does not show improvements in detection of changes during the change detection task, closer estimates of human figure presentations in the numerosity task, reduction in perception of faces and objects in the pareidolia task, and/or improvements in working memory capacity in the visual n-back test, subsequent FUS neuromodulation parameters are adjusted by increasing the sonication energy until reaching the maximum level for the specific device for the total 30 duration. For the 220 kHz system, the initial sonication block starts with a FUS neuromodulation dose of 50 W for 5 min and if there are no clinically relevant changes in visual spatial assessments then 60 W is used for the second 5 min block. If there are still no clinically relevant improvements, then 70 W is used for the third 5 min block. If there are still no clinically relevant improvements, then 80 W is used for the fourth block. If still yet no clinically relevant improvements occur, then 90 W is used for the fifth block. If there are still no clinically relevant improvements, then 100 W is used.

### III. Results

The application of FUS to the putamen results in an immediate and measurable improvement in task performance compared to baseline and sham conditions.

Specifically, the patient shows:
1. Improvements in detection of changes during the change detection task
2. Closer estimates of human figure presentations in the numerosity task
3. Reduction in perception of faces and objects in the pareidolia task
4. Improvements in working memory capacity in the visual n-back task

These results demonstrate that FUS neuromodulation of the putamen can enhance the processing of visuospatial information, thereby bypassing or compensating for visuospatial deficits from Parkinson's disease.

### Example IV

### I. Patient Preparation and Targeting

Pre-operative MRI imaging is used to identify the stereotactic coordinates of the nucleus accumbens.

### II. Methodology and Stimuli

The patient is positioned before a display screen. A baseline assessment is conducted where the patient is presented with a battery of visual stimuli designed to test:
Visual working memory, attentional filtering, and visual transition: Visual change detection test. A visual change detection test is a cognitive task where the patient identifies differences between two similar visual scenes or stimuli to measure visual working memory capacity. A specific method includes a “flicker paradigm,” which uses brief disruptions between identical images to reveal missed changes, and “one-shot” tasks where a single probe is compared to an initial display, revealing how many items can be held in short-term memory.
Numerosity and Visual salience: Human figure vs non-human figure numerosity task. A human figure vs. non-human figure numerosity task compares how easily people estimate the number of people (humans) versus objects.
Visuoperceptual interference: Pareidolia test. A pareidolia test is a neuropsychological tool using ambiguous visual noise designed to measure how often the patient perceives meaningful patterns (like faces) in randomness by detecting heightened illusory responses that signal visual hallucinations. While normal pareidolia is common (seeing faces in clouds), the test quantifies abnormal levels, distinguishing between healthy individuals and a patient with Parkinson where such illusions ca be more frequent and intense.
Visual Working memory: Visual object N-Back. A visual object N-back is a cognitive test measuring working memory by showing a sequence of different objects (shapes, pictures) and asking the patient to identify if the current object matches the one shown ‘n’ trials (e.g., 1-back, 2-back) earlier in the sequence, training the ability to hold and update visual information, crucial for attention, learning, and cognitive flexibility.

| Cognitive Construct | | |
|---|---|---|
| Being Assessed | Task | Metric Detected |
| I. Capacity | Change | Visual Working Memory |
| | Detection | (VWM) |
| II. Precision | Numerosity | Approximate Number |
| | | System (ANS) |
| III. Accuracy | Pareidolia | Noise-Signal Discrimination |
| IV. Fluidity | Visual n-Back | Working Memory Updating |

After the baseline assessments, initial FUS neuromodulation is delivered while the patient carries out one or more of the tasks listed above. Parameters for initial FUS neuromodulation are: for a frequency of 220 kilohertz (kHz); a power of 50-100 watts (W); a duty cycle of 3.3% per subspot; 100 repetitions; a repetition time (TR)=3 seconds; a pulse duration of 100 milliseconds (ms); for up to 30 minutes (mins) in six 5 min sonication blocks for one treatment (this equates to estimated root mean square pressure of in situ peak rarefactional pressure of 1.3-2.4 megapascal (MPa)). For a system with a frequency of 500 kHz; a pulse rate of 0.33; a duty cycle of 1.67%; pulse duration=50 ms; for up to 30 mins in six 5 min sonication blocks for one treatment (this equates to in situ peak rarefactional pressure of 1.27-2.2 MPa). Once the initial FUS neuromodulation sonication block is over, the patient carries out identical task assessment as the baseline task assessment. FUS neuromodulation parameters are adjusted based on the comparison of post-sonication block task assessment and the baseline task assessment. If the patient shows improvements in detection of changes during the change detection task, closer estimates of human figure presentations in the numerosity task, reduction in perception of faces and objects in the pareidolia task, and/or improvements in working memory capacity in the visual n-back test, the patient receives additional FUS neuromodulation sonication sessions up to 30 min of total sonication (six 5 min blocks) with the same neuromodulation parameters. If the patient does not show improvements in detection of changes during the change detection task, closer estimates of human figure presentations in the numerosity task, reduction in perception of faces and objects in the pareidolia task, and/or improvements in working memory capacity in the visual n-back test, subsequent FUS neuromodulation parameters are adjusted by increasing the sonication energy until reaching the maximum level for the specific device for the total 30 duration. For the 220 kHz system, the initial sonication block starts with a FUS neuromodulation dose of 50 W for 5 min and if there are no clinically relevant changes in visual spatial assessments then 60 W is used for the second 5 min block. If there are still no clinically relevant improvements, then 70 W is used for the third 5 min block. If there are still no clinically relevant improvements, then 80 W is used for the fourth block. If still yet no clinically relevant improvements occur, then 90 W is used for the fifth block. If there are still no clinically relevant improvements, then 100 W is used.

### III. Results

The application of FUS to the nucleus accumbens results in an immediate and measurable improvement in task performance compared to baseline and sham conditions.

Specifically, the patient shows:
1. Improvements in detection of changes during the change detection task
2. Closer estimates of human figure presentations in the numerosity task
3. Reduction in perception of faces and objects in the pareidolia task
4. Improvements in working memory capacity in the visual n-back task

These results demonstrate that FUS neuromodulation of the nucleus accumbens can enhance the processing of visuospatial information, thereby bypassing or compensating for visuospatial deficits from Parkinson's disease.

### Example V

### I. Patient Preparation and Targeting

Pre-operative MRI imaging is used to identify the stereotactic coordinates of the ventral striatum.

### II. Methodology and Stimuli

The patient is positioned before a display screen. A baseline assessment is conducted where the patient is presented with a battery of visual stimuli designed to test:
Visual working memory, attentional filtering, and visual transition: Visual change detection test. A visual change detection test is a cognitive task where the patient identifies differences between two similar visual scenes or stimuli to measure visual working memory capacity. A specific method includes a “flicker paradigm,” which uses brief disruptions between identical images to reveal missed changes, and “one-shot” tasks where a single probe is compared to an initial display, revealing how many items can be held in short-term memory.
Numerosity and Visual salience: Human figure vs non-human figure numerosity task. A human figure vs. non-human figure numerosity task compares how easily people estimate the number of people (humans) versus objects.
Visuoperceptual interference: Pareidolia test. A pareidolia test is a neuropsychological tool using ambiguous visual noise designed to measure how often the patient perceives meaningful patterns (like faces) in randomness by detecting heightened illusory responses that signal visual hallucinations. While normal pareidolia is common (seeing faces in clouds), the test quantifies abnormal levels, distinguishing between healthy individuals and a patient with Parkinson where such illusions ca be more frequent and intense.
Visual Working memory: Visual object N-Back. A visual object N-back is a cognitive test measuring working memory by showing a sequence of different objects (shapes, pictures) and asking the patient to identify if the current object matches the one shown ‘n’ trials (e.g., 1-back, 2-back) earlier in the sequence, training the ability to hold and update visual information, crucial for attention, learning, and cognitive flexibility.

| Cognitive Construct | | |
|---|---|---|
| Being Assessed | Task | Metric Detected |
| I. Capacity | Change | Visual Working Memory |
| | Detection | (VWM) |
| II. Precision | Numerosity | Approximate Number |
| | | System (ANS) |
| III. Accuracy | Pareidolia | Noise-Signal Discrimination |
| IV. Fluidity | Visual n-Back | Working Memory Updating |

After the baseline assessments, initial FUS neuromodulation is delivered while the patient carries out one or more of the tasks listed above. Parameters for initial FUS neuromodulation are: for a frequency of 220 kilohertz (kHz); a power of 50-100 watts (W); a duty cycle of 3.3% per subspot; 100 repetitions; a repetition time (TR)=3 seconds; a pulse duration of 100 milliseconds (ms); for up to 30 minutes (mins) in six 5 min sonication blocks for one treatment (this equates to estimated root mean square pressure of in situ peak rarefactional pressure of 1.3-2.4 megapascal (MPa)). For a system with a frequency of 500 kHz; a pulse rate of 0.33; a duty cycle of 1.67%; pulse duration=50 ms; for up to 30 mins in six 5 min sonication blocks for one treatment (this equates to in situ peak rarefactional pressure of 1.27-2.2 MPa). Once the initial FUS neuromodulation sonication block is over, the patient carries out identical task assessment as the baseline task assessment. FUS neuromodulation parameters are adjusted based on the comparison of post-sonication block task assessment and the baseline task assessment. If the patient shows improvements in detection of changes during the change detection task, closer estimates of human figure presentations in the numerosity task, reduction in perception of faces and objects in the pareidolia task, and/or improvements in working memory capacity in the visual n-back test, the patient receives additional FUS neuromodulation sonication sessions up to 30 min of total sonication (six 5 min blocks) with the same neuromodulation parameters. If the patient does not show improvements in detection of changes during the change detection task, closer estimates of human figure presentations in the numerosity task, reduction in perception of faces and objects in the pareidolia task, and/or improvements in working memory capacity in the visual n-back test, subsequent FUS neuromodulation parameters are adjusted by increasing the sonication energy until reaching the maximum level for the specific device for the total 30 duration. For the 220 kHz system, the initial sonication block starts with a FUS neuromodulation dose of 50 W for 5 min and if there are no clinically relevant changes in visual spatial assessments then 60 W is used for the second 5 min block. If there are still no clinically relevant improvements, then 70 W is used for the third 5 min block. If there are still no clinically relevant improvements, then 80 W is used for the fourth block. If still yet no clinically relevant improvements occur, then 90 W is used for the fifth block. If there are still no clinically relevant improvements, then 100 W is used.

### III. Results

The application of FUS to the ventral striatum results in an immediate and measurable improvement in task performance compared to baseline and sham conditions.

Specifically, the patient shows:
1. Improvements in detection of changes during the change detection task
2. Closer estimates of human figure presentations in the numerosity task
3. Reduction in perception of faces and objects in the pareidolia task
4. Improvements in working memory capacity in the visual n-back task

These results demonstrate that FUS neuromodulation of the ventral striatum can enhance processing of visuospatial information, thereby bypassing or compensating for visuospatial deficits from Parkinson's disease.

### Example VI

### The Ventral Internal Capsule

### I. Patient Preparation and Targeting

Pre-operative MRI imaging is used to identify the stereotactic coordinates of the ventral internal capsule.

### II. Methodology and Stimuli

The patient is positioned before a display screen. A baseline assessment is conducted where the patient is presented with a battery of visual stimuli designed to test:
Visual working memory, attentional filtering, and visual transition: Visual change detection test. A visual change detection test is a cognitive task where the patient identifies differences between two similar visual scenes or stimuli to measure visual working memory capacity. A specific method includes a “flicker paradigm,” which uses brief disruptions between identical images to reveal missed changes, and “one-shot” tasks where a single probe is compared to an initial display, revealing how many items can be held in short-term memory.
Numerosity and Visual salience: Human figure vs non-human figure numerosity task. A human figure vs. non-human figure numerosity task compares how easily people estimate the number of people (humans) versus objects.
Visuoperceptual interference: Pareidolia test. A pareidolia test is a neuropsychological tool using ambiguous visual noise designed to measure how often the patient perceives meaningful patterns (like faces) in randomness by detecting heightened illusory responses that signal visual hallucinations. While normal pareidolia is common (seeing faces in clouds), the test quantifies abnormal levels, distinguishing between healthy individuals and a patient with Parkinson where such illusions ca be more frequent and intense.
Visual Working memory: Visual object N-Back. A visual object N-back is a cognitive test measuring working memory by showing a sequence of different objects (shapes, pictures) and asking the patient to identify if the current object matches the one shown ‘n’ trials (e.g., 1-back, 2-back) earlier in the sequence, training the ability to hold and update visual information, crucial for attention, learning, and cognitive flexibility.

| Cognitive Construct | | |
|---|---|---|
| Being Assessed | Task | Metric Detected |
| I. Capacity | Change | Visual Working Memory |
| | Detection | (VWM) |
| II. Precision | Numerosity | Approximate Number |
| | | System (ANS) |
| III. Accuracy | Pareidolia | Noise-Signal Discrimination |
| IV. Fluidity | Visual n-Back | Working Memory Updating |

After the baseline assessments, initial FUS neuromodulation is delivered while the patient carries out one or more of the tasks listed above. Parameters for initial FUS neuromodulation are: for a frequency of 220 kilohertz (kHz); a power of 50-100 watts (W); a duty cycle of 3.3% per subspot; 100 repetitions; a repetition time (TR)=3 seconds; a pulse duration of 100 milliseconds (ms); for up to 30 minutes (mins) in six 5 min sonication blocks for one treatment (this equates to estimated root mean square pressure of in situ peak rarefactional pressure of 1.3-2.4 megapascal (MPa)). For a system with a frequency of 500 kHz; a pulse rate of 0.33; a duty cycle of 1.67%; pulse duration=50 ms; for up to 30 mins in six 5 min sonication blocks for one treatment (this equates to in situ peak rarefactional pressure of 1.27-2.2 MPa). Once the initial FUS neuromodulation sonication block is over, the patient carries out identical task assessment as the baseline task assessment. FUS neuromodulation parameters are adjusted based on the comparison of post-sonication block task assessment and the baseline task assessment. If the patient shows improvements in detection of changes during the change detection task, closer estimates of human figure presentations in the numerosity task, reduction in perception of faces and objects in the pareidolia task, and/or improvements in working memory capacity in the visual n-back test, the patient receives additional FUS neuromodulation sonication sessions up to 30 min of total sonication (six 5 min blocks) with the same neuromodulation parameters. If the patient does not show improvements in detection of changes during the change detection task, closer estimates of human figure presentations in the numerosity task, reduction in perception of faces and objects in the pareidolia task, and/or improvements in working memory capacity in the visual n-back test, subsequent FUS neuromodulation parameters are adjusted by increasing the sonication energy until reaching the maximum level for the specific device for the total 30 duration. For the 220 kHz system, the initial sonication block starts with a FUS neuromodulation dose of 50 W for 5 min and if there are no clinically relevant changes in visual spatial assessments then 60 W is used for the second 5 min block. If there are still no clinically relevant improvements, then 70 W is used for the third 5 min block. If there are still no clinically relevant improvements, then 80 W is used for the fourth block. If still yet no clinically relevant improvements occur, then 90 W is used for the fifth block. If there are still no clinically relevant improvements, then 100 W is used.

### III. Results

The application of FUS to the ventral internal capsule results in an immediate and measurable improvement in task performance compared to baseline and sham conditions.

Specifically, the patient shows:
1. Improvements in detection of changes during the change detection task
2. Closer estimates of human figure presentations in the numerosity task
3. Reduction in perception of faces and objects in the pareidolia task
4. Improvements in working memory capacity in the visual n-back task

These results demonstrate that FUS neuromodulation of the ventral internal capsule can enhance the processing of visuospatial information, thereby bypassing or compensating for visuospatial deficits from Parkinson's disease.

### Example VII

### I. Patient Preparation and Targeting

Pre-operative MRI imaging is used to identify the stereotactic coordinates of a thalamic relay nucleus such as the medial geniculate nucleus (MGN)

### II. Methodology and Stimuli

The patient is positioned before a display screen. A baseline assessment is conducted where the patient is presented with a battery of visual stimuli designed to test:
Visual working memory, attentional filtering, and visual transition: Visual change detection test. A visual change detection test is a cognitive task where the patient identifies differences between two similar visual scenes or stimuli to measure visual working memory capacity. A specific method includes a “flicker paradigm,” which uses brief disruptions between identical images to reveal missed changes, and “one-shot” tasks where a single probe is compared to an initial display, revealing how many items can be held in short-term memory.
Numerosity and Visual salience: Human figure vs non-human figure numerosity task. A human figure vs. non-human figure numerosity task compares how easily people estimate the number of people (humans) versus objects.
Visuoperceptual interference: Pareidolia test. A pareidolia test is a neuropsychological tool using ambiguous visual noise designed to measure how often the patient perceives meaningful patterns (like faces) in randomness by detecting heightened illusory responses that signal visual hallucinations. While normal pareidolia is common (seeing faces in clouds), the test quantifies abnormal levels, distinguishing between healthy individuals and a patient with Parkinson where such illusions ca be more frequent and intense.
Visual Working memory: Visual object N-Back. A visual object N-back is a cognitive test measuring working memory by showing a sequence of different objects (shapes, pictures) and asking the patient to identify if the current object matches the one shown ‘n’ trials (e.g., 1-back, 2-back) earlier in the sequence, training the ability to hold and update visual information, crucial for attention, learning, and cognitive flexibility.

| Cognitive Construct | | |
|---|---|---|
| Being Assessed | Task | Metric Detected |
| I. Capacity | Change | Visual Working Memory |
| | Detection | (VWM) |
| II. Precision | Numerosity | Approximate Number |
| | | System (ANS) |
| III. Accuracy | Pareidolia | Noise-Signal Discrimination |
| IV. Fluidity | Visual n-Back | Working Memory Updating |

After the baseline assessments, initial FUS neuromodulation is delivered while the patient carries out one or more of the tasks listed above. Parameters for initial FUS neuromodulation are: for a frequency of 220 kilohertz (kHz); a power of 50-100 watts (W); a duty cycle of 3.3% per subspot; 100 repetitions; a repetition time (TR)=3 seconds; a pulse duration of 100 milliseconds (ms); for up to 30 minutes (mins) in six 5 min sonication blocks for one treatment (this equates to estimated root mean square pressure of in situ peak rarefactional pressure of 1.3-2.4 megapascal (MPa)). For a system with a frequency of 500 kHz; a pulse rate of 0.33; a duty cycle of 1.67%; pulse duration=50 ms; for up to 30 mins in six 5 min sonication blocks for one treatment (this equates to in situ peak rarefactional pressure of 1.27-2.2 MPa). Once the initial FUS neuromodulation sonication block is over, the patient carries out identical task assessment as the baseline task assessment. FUS neuromodulation parameters are adjusted based on the comparison of post-sonication block task assessment and the baseline task assessment. If the patient shows improvements in detection of changes during the change detection task, closer estimates of human figure presentations in the numerosity task, reduction in perception of faces and objects in the pareidolia task, and/or improvements in working memory capacity in the visual n-back test, the patient receives additional FUS neuromodulation sonication sessions up to 30 min of total sonication (six 5 min blocks) with the same neuromodulation parameters. If the patient does not show improvements in detection of changes during the change detection task, closer estimates of human figure presentations in the numerosity task, reduction in perception of faces and objects in the pareidolia task, and/or improvements in working memory capacity in the visual n-back test, subsequent FUS neuromodulation parameters are adjusted by increasing the sonication energy until reaching the maximum level for the specific device for the total 30 duration. For the 220 kHz system, the initial sonication block starts with a FUS neuromodulation dose of 50 W for 5 min and if there are no clinically relevant changes in visual spatial assessments then 60 W is used for the second 5 min block. If there are still no clinically relevant improvements, then 70 W is used for the third 5 min block. If there are still no clinically relevant improvements, then 80 W is used for the fourth block. If still yet no clinically relevant improvements occur, then 90 W is used for the fifth block. If there are still no clinically relevant improvements, then 100 W is used.

### III. Results

The application of FUS to a thalamic relay nucleus results in an immediate and measurable improvement in task performance compared to baseline and sham conditions.

Specifically, the patient shows:
1. Improvements in detection of changes during the change detection task
2. Closer estimates of human figure presentations in the numerosity task
3. Reduction in perception of faces and objects in the pareidolia task
4. Improvements in working memory capacity in the visual n-back task

These results demonstrate that FUS neuromodulation of a thalamic relay nucleus can enhance the processing of visuospatial information, thereby bypassing or compensating for visuospatial deficits from Parkinson's disease.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What is claimed is:

1. A method of improving a visuospatial impairment in a patient suffering from Parkinson's Disease comprising:
delivering a focused ultrasound signal to a neural target site of the patient; and
improving the patient's visuospatial impairment.

2. The method of claim 1, wherein improving the visuospatial impairment comprising improving a visual perception, a blurry vision, a judging distance, a depth perception, another visual and spatial deficit, or combinations thereof.

3. The method of claim 1, wherein the neural target site comprises a subthalamic nucleus, a globus pallidus, a putamen, a nucleus accumbens, a ventral striatum, a ventral internal capsule, a thalamic relay nucleus, or combinations thereof.

4. The method of claim 1, further comprising asking the patient to perform a task reliant on depth perception prior to delivering the focused ultrasound signal.
